(19) United States
(12) Patent Application Publication
(10) Pub. No.: US 2026/0256798 A1
(43) Pub. Date: Sep. 3, 2026

(54) COMBINATIONS OF A SERD FOR THE TREATMENT OF CANCER

(71) Applicant: AstraZeneca AB, Södertälje (SE)

(72) Inventors: Larissa DE SOUZA CARNEVALLI, Cambridge (GB); Simon Thomas BARRY, Cambridge (GB)

(21) Appl. No.: 19/119,802

(22) PCT Filed: Oct. 16, 2023
(86) PCT No.: PCT/EP2023/078625

§ 371 Date: Apr. 10, 2025

Related U.S. Application Data
(60) Provisional application No. 63/379,835, filed on Oct. 17, 2022.
(60) Provisional application No. 63/487,744, filed on Mar. 1, 2023.

(51) Int. Cl. A61K 31/5377; A61K 31/517

(52) U.S. Cl. CPC A61K 31/5377; A61K 31/517

(57) ABSTRACT

The present specification relates a method of treating a patient with hormone receptor positive (HR+), human epidermal growth factor receptor 2 negative (HER2−), breast cancer comprising administering an oral selective estrogen receptor degrader (SERD) in combination with an AKT inhibitor or an mTOR inhibitor and/or a cyclin dependent kinase 4/6 inhibitor (CDK4/6i) to the patient in need thereof.

### CROSS-REFERENCE TO RELATED APPLICATIONS

This application is U.S. National Stage application of International Application No. PCT/EP2023/078625, filed on Oct. 16, 2023, which claims benefit under 35 U.S.C. § 119 (e) of the U.S. Provisional Applications No. 63/487,744, filed on Mar. 1, 2023 and 63/379,835, filed on Oct. 17, 2022. Each of the above listed applications is incorporated by reference herein in its entirety for all purposes.

### FIELD

The specification relates to the use of oral SERDs in combination with AKT or mTOR and/or CDK4/6 inhibitors to treat cancer, for example breast cancer.

### BACKGROUND

Next-generation oral selective estrogen receptor degraders (ngSERDs) are aiming to become the backbone endocrine therapy (ET) for patients with estrogen receptor (ER) positive breast cancer by delivering greater ER signalling blockade than current therapies and tackling key mechanisms of resistance. Camizestrant (AZD9833) is an ngSERD for the treatment of ER+ breast cancer which has demonstrated selective ERα degradation, pure ER antagonism and significant anti-tumour activity in ESR1 wild-type (ESR1 wt) and mutant (ESR1m) tumors, as well as encouraging clinical activity in early phase clinical trials.

ER+ breast cancer is responsive to therapies targeting ERα and CDK4/6 signalling in the adjuvant and metastatic settings. To explore the potential of SERDs as a backbone ET therapy, camizestrant was partnered with either palbociclib or abemaciclib in CDK4/6 inhibitor naïve and resistant in-vitro and in-vivo models, reflecting the SERENA-1 and SERENA-4 clinical trials. Combination benefit was observed in-vitro in 3 parental ER+ breast cancer cell lines, moreover camizestrant plus abemaciclib showed activity in palbociclib-resistant cell lines including lines harbouring CCNE1amp and RB1 loss. In-vivo, combination of camizestrant and palbociclib or abemaciclib was well tolerated with the combinations promoting improved efficacy in ESR1 wt and ESR1m PDX (“patient-derived xenograft”) tumour models relative to the camizestrant and CDK4/6 inhibitor monotherapy arms in the study.

ER+ positive breast cancer has a high prevalence of alterations in the PI3K/AKT/PTEN pathway which provides opportunities for treatment with PI3K/AKT pathway inhibitors. The ngSERD camizestrant delivered enhanced efficacy when combined with mTOR inhibitor everolimus and AKT inhibitor capivasertib in CTC-174, an ESR1m and a PI3KCAm tumor model (i.e. a model in which both the estrogen receptor and PI3KCA carry a mutation). Additionally, combination with the AKT inhibitor capivasertib was more efficacious than single treatments at clinically relevant dose and schedules in both PI3K wt and mutated pathway in PDX models. These data demonstrate the interplay between PI3K pathway inhibition and camizestrant's mechanism of action. Notably, the combinations of camizestrant and capivasertib proved significantly more active than optimal doses of fulvestrant and capivasertib in both ESR1m and ESR1 wt PDX models, suggesting that the clinical benefit may be derivable from use of camizestrant in preference to fulvestrant in combination with an AKT inhibitor.

Finally, this specification also discloses the use of a triple combination of camizestrant, capivasertib and palbociclib across palbociclib-resistant models representative of PI3KCA/AKT wt and mutated tumors.

This strategy delivered robust efficacy across these models comparing to single treatments and double combinations. The camizestrant, capivasertib and palbociclib triplet led to durable regressions at clinically achievable doses of all compounds, irrespective of genetic background. These preclinical data demonstrate the potential of SERDs like camizestrant to become the backbone ET, with high combinability in-vivo with CDK4/6, mTOR and AKT inhibitors. The activity profile of these combinations reveal an opportunity to impact the care of early and metastatic ER+ breast cancer patients by delivering benefit across broad patient populations, including those with ESR1 wt or ESR1m tumours, that is independent of PI3K pathway mutation status, and is exhibited in both CDK4/6i naïve and refractory patients.

### SUMMARY

The present specification provides a means for enhancing the anti-proliferative effects of SERD treatment in cancer (for example breast cancer) by utilising SERDs (for example ngSERDs) in combination with mTOR, AKT and/or CDK4/6 inhibitors.

In an aspect, there is provided a SERD for use in the treatment of cancer, where the SERD is administered in combination with an AKT inhibitor or an mTOR inhibitor, and/or a CDK4/6 inhibitor.

In an aspect, there is provided a SERD for use in the treatment of cancer, where the SERD is administered in combination with:
an AKT inhibitor or an mTOR inhibitor;
a CDK4/6 inhibitor; or
an AKT inhibitor or an mTOR inhibitor and a CDK4/6 inhibitor.

The terms “treat,” “treating,” and “treatment” refer to at least partially alleviating, inhibiting, preventing and/or ameliorating a condition, disorder, or disease, such as breast cancer. The term “treatment of cancer” includes both in vitro and in-vivo treatments, including in warm-blooded animals such as humans. The effectiveness of treatment of cancer can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging survival.

Treatments can also be assessed with regard to the nature and extent of side effects associated with the treatment. Furthermore, effectiveness can be assessed with regard to biomarkers, such as levels of expression or phosphorylation of proteins known to be associated with particular biological phenomena.

Other assessments of effectiveness are known to those of skill in the art.

The phrase “in combination with” and similar terms encompass administration of two or more active pharmaceutical ingredients to a subject and include simultaneous administration in separate compositions, administration at different times in separate compositions, or administration in a composition in which two or more active pharmaceutical ingredients are present. For triplet combinations, hybrid administrations where two drugs are administered simultaneously and one drug separately or sequentially to the others are encompassed by the preceding definitions.

In embodiments administration of the SERD and each inhibitor is separate, sequential, or simultaneous.

In embodiments administration of the SERD and each inhibitor is separate.

In embodiments administration of the SERD and each inhibitor is sequential.

In embodiments administration of the SERD and each inhibitor is simultaneous.

In a further aspect, there is provided the use of a SERD in the manufacture of a medicament for the treatment of cancer, where the SERD is administered in combination with an AKT inhibitor or an mTOR inhibitor, and/or a CDK4/6 inhibitor.

In a further aspect, there is provided a method of treating cancer in an animal patient in need of such treatment, comprising administering to the animal patient a therapeutically effective amount of a SERD, where the SERD is administered in combination with a therapeutically effective amount of an AKT inhibitor or an mTOR inhibitor, and/or a CDK4/6 inhibitor.

The term “therapeutically effective amount” refers to that amount of a compound or combination of compounds as described herein that is sufficient to effect the intended application including, but not limited to, disease treatment. A therapeutically effective amount may vary depending upon the intended application (in vitro or in-vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g. the amount of apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

In a further aspect, there is provided a method of treating cancer in an animal patient in need of such treatment, comprising administering to the animal patient a therapeutically effective amount of a SERD, where the SERD is administered in combination with an AKT inhibitor or an mTOR inhibitor and a CDK4/6 inhibitor.

In a further aspect, there is provided a method of treating cancer in an animal patient in need of such treatment, comprising administering to the animal patient a first amount of a SERD, a second amount of an AKT inhibitor or an mTOR inhibitor, and a third amount of a CDK4/6 inhibitor, where the first amount, the second amount and the third amount together comprise a therapeutically effective amount.

In a further aspect, there is provided a pharmaceutical composition comprising a SERD in combination with an AKT inhibitor or an mTOR inhibitor, and/or a CDK4/6 inhibitor, and a pharmaceutically acceptable excipient.

The term “pharmaceutically acceptable” is used to specify that an object (for example a salt, dosage form [such as a tablet or capsule] or excipient [such as a diluent or carrier]) is suitable for use in patients. An example list of pharmaceutically acceptable salts can be found in the “Handbook of Pharmaceutical Salts: Properties, Selection and Use”, P. H. Stahl and C. G. Wermuth, editors, Weinheim/Zurich: Wiley-VCH/VFiCA, 2002 or subsequent editions.

Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminium. Organic bases from which salts can be derived include, for example, primary, secondary, and tertiary amines, substituted amines including naturally occurring substituted amines, cyclic amines and basic ion exchange resins. Examples include isopropylamine, trimethylamine, diethylamine, triethylamine, tripropylamine, and ethanolamine.

### LIST OF FIGURES

FIG. 1: Mouse patient-derived xenograft (PDX) assay. Combination of AZD9833 at 10 mg/kg, everolimus at 5 mg/kg were dosed once daily via oral administration (PO) for 28 days at a volume of 0.1 ml per 10 g of mouse delivers enhanced efficacy compared to monotherapy in D538G ESR1mt/PI3KCA N345K PDX CTC174. Statistical analysis was performed by one tailed, unequal variance t-test versus log (change in tumor volume) compared with vehicle control at the final day of treatment. ****p<0.0001.

FIG. 2: Mouse patient-derived xenograft assay. Combination of AZD9833 and capivasertib, at 10 mg/kg and 85 mg/kg, respectively, dosed via oral administration (PO) for 28 days, wherein camizestrant was dosed continuously and capivasertib was dosed on a 4 days on, 3 days off schedule, delivers enhanced efficacy compared to AZD9833 or capivasertib monotherapy in D538G ESR1mt/PI3KCA N345K PDX CTC174. Statistical analysis was performed by one tailed, unequal variance t-test versus log (change in tumor volume) compared with vehicle control at the final day of treatment. ****p<0.0001.

FIG. 3: Mouse patient-derived xenograft assay. 10 mg/kg AZD9833, 50 mg/kg palbociclib or the combination of both agents at the same doses, administered once daily by oral gavage throughout the study. AZD9833 in combination with the CDK4/6 inhibitor palbociclib delivers enhanced efficacy compared to monotherapy in D538G ESR1mt/PI3KCA N345K PDX CTC174. Statistical analysis was performed by one tailed, unequal variance t-test versus log (change in tumor volume) compared with vehicle control at the final day of treatment. *p<0.05, **p<0.01, ***p<0.001, ****p<0.0001.

FIG. 4: Palbociclib resistant cell lines characterization. Genetic alteration assessed by whole exosome sequencing (WES) of cell lines indicated in table. Combination matrix plots showing a seven day cell viability assays for combination treatments with camizestrant and palbociclib or abemaciclib in MCF7 parental and palbociclib-resistant cell lines (PC1, PC6, PC8) were measured using Cell Titer Glo (CTG, Promega). Cells were seeded in 60 μl of medium one day prior to treatment. The next day (day 0) assay plates were dosed with compounds and read on day 7. An untreated plate was read on day 0. These results were obtained with the addition of 30 μl of CTG and a read for luminescence after a 30 minute incubation period at ambient temperature. The data was normalized to the original day 0 seeding and the maximum day 7 growth (DMSO only).

FIG. 5: Mouse patient-derived xenograft assay. AZD9833 combination with CDK4/6 and/or mTOR/AKT inhibitors promotes robust activity in PDX models. In vivo combination of camizestrant at 10 mg/kg daily with palbociclib 50 mg/kg, abemaciclib 50 mg/kg daily and capivasertib 130 mg/kg in PDX ST1799 dosed for 40 days (grey area). Note: graph was subdivided in two subgraphs due to large number of treatment arms, vehicle and palbociclib arms are the same in the two subgraphs plotted. Statistical analysis was performed by one tailed, unequal variance t-test versus log (change in tumor volume) compared with vehicle control at the final day of treatment. Statistical analysis was performed by one tailed, unequal variance t-test versus log (change in tumor volume) compared with vehicle control at the final day of treatment. *p<0.05, **p<0.01, ***p<0.001, ****p<0.0001.

FIG. 6: Mouse patient-derived xenograft in ST3632 model an ESR1 wt model. AZD9833 combination with CDK4/6 inhibitors promotes robust activity in ER+ PDX models derived from a primary tumor, representative of early disease and insensitive to palbociclib monotherapy. AZD9833 dosed at 10 mg/kg daily with palbociclib 50 mg/kg, abemaciclib 50 mg/kg daily orally. Statistical analysis was performed by one tailed, unequal variance t-test versus log (change in tumor volume) compared with vehicle control at the final day of treatment. Statistical analysis was performed by one tailed, unequal variance t-test versus log (change in tumor volume) compared with vehicle control at the final day of treatment. *p<0.05, **p<0.01, ***p<0.001, ****p<0.0001.

FIG. 7: Combination efficacy of AZ9833, palbociclib and capivasertib triplet compared to combination of fulvestrant and palbociclib. Patient-derived xenograft preclinical models were treated with palbociclib and fulvestrant and with a triplet combination of camizestrant, capivasertib and palbociclib in PDX tumor models. The triplet treatment with combined ER, CDK4/6 and AKT inhibition was superior than palbociclib/fulvestrant combination and broadly effective in both PI3K pathway mutated and WT PDX models. AZD9833 was dosed at 10 mg/kg daily with palbociclib 50 mg/kg daily, capivasertib 130 mg/kg 4 days on, 3 days off. Statistical analysis was performed by one tailed, unequal variance t-test versus log (change in tumor volume) compared with vehicle control at the final day of treatment. *p<0.05, **p<0.01, ***p<0.001, ****p<0.0001.

FIG. 8. Summary of model characterization and combination efficacy of AZ9833, palbociclib and capivasertib triplet. Model characteristics of patient-derived xenograft preclinical models are summarised in heatmap. Triplet activity combination of AZD9833, capivasertib and palbociclib compared to monotherapy and doublet arm triplet. The triplet treatment with combined ER, CDK4/6 and AKT inhibition was superior than palbociclib/fulvestrant combination and broadly effective in both PI3K pathway mutated and wild type PDX models. Statistical analysis was performed by one tailed, unequal variance t-test versus log (change in tumor volume) compared with vehicle control at the final day of treatment. Statistical analysis was performed by one tailed, unequal variance t-test versus log (change in tumor volume) compared with vehicle control at the final day of treatment. *p<0.05, **p<0.01, ***p<0.001, ****p<0.0001.

FIG. 9. Combination efficacy of AZD9833 doublet and triplet combinations with capivasertib and/or palbociclib in an ST1799 model (ESR1 wt, PI3KCAm E542K). Patient-derived xenograft preclinical models were used to compare a triplet combination of AZD9833, capivasertib and palbociclib with various corresponding monotherapy and doublet combinations (fulvestrant+palbociclib, fulvestrant+capivasertib, camizestrant+palbociclib and camizestrant+capivasertib). FIG. 9 shows tumor volume plots for ER+ breast cancer PDX in a 28-day efficacy study, displaying all treatment arms. Dosing: oral palbociclib 50 mg/kg once daily, subcutaneous fulvestrant 5 mg/kg weekly, oral camizestrant 10 mg/kg once daily, capivasertib 130 mg/kg twice daily on a 4 days on, 3 days off dosing schedule. The triplet combination was superior to doublet combinations and monotherapy. The combination of camizestrant and capivasertib was also superior to the combination of fulvestrant and capivasertib.

FIG. 10. Combination efficacy of AZD9833 doublet and triplet combinations with capivasertib and/or palbociclib in an ST3632 model (ESR1 wt, AKT1m E17K). Patient-derived xenograft preclinical models were used to compare a triplet combination of AZD9833, capivasertib and palbociclib with various corresponding monotherapy and doublet combinations (fulvestrant+palbociclib, fulvestrant+capivasertib, camizestrant+palbociclib and camizestrant+capivasertib). FIG. 10 shows tumor volume plots for ER+ breast cancer PDX in a 28-day efficacy study, displaying all treatment arms. Dosing: oral palbociclib 50 mg/kg once daily, subcutaneous fulvestrant 5 mg/kg weekly, oral camizestrant 10 mg/kg once daily, capivasertib 130 mg/kg twice daily on a 4 days on, 3 days off dosing schedule. The triplet combination was superior to doublet combinations and monotherapy. The combination of camizestrant and capivasertib was also superior to the combination of fulvestrant and capivasertib.

FIG. 11. Combination efficacy of AZD9833 doublet and triplet combinations with capivasertib and/or palbociclib in an ST941 model (ESR1m Y537S). Patient-derived xenograft preclinical models were used to compare a triplet combination of AZD9833, capivasertib and palbociclib with various corresponding monotherapy and doublet combinations (fulvestrant+palbociclib, fulvestrant+capivasertib, camizestrant+palbociclib and camizestrant+capivasertib). FIG. 11 shows tumor volume plots for ER+ breast cancer PDX in a 28-day efficacy study, displaying all treatment arms. Dosing: oral palbociclib 50 mg/kg once daily, subcutaneous fulvestrant 5 mg/kg weekly, oral camizestrant 10 mg/kg once daily, capivasertib 130 mg/kg twice daily on a 4 days on, 3 days off dosing schedule. The triplet combination was superior to doublet combinations and monotherapy. The combination of camizestrant and capivasertib was also superior to the combination of fulvestrant and capivasertib.

FIG. 12. Combination efficacy of AZD9833 doublet and triplet combinations with capivasertib and/or palbociclib in a CTC174 model (altered PI3KCA/AKT or PTEN ER+; ESR1m D538G, PI3KCAm N345K). Xenograft preclinical models were used to compare a triplet combination of AZD9833, capivasertib and palbociclib with various corresponding monotherapy and doublet combinations (fulvestrant+palbociclib, fulvestrant+capivasertib, camizestrant+palbociclib and camizestrant+capivasertib). FIG. 12 shows tumor volume plots for ER+ breast cancer PDX in a 28-day efficacy study, displaying all treatment arms. Dosing: oral palbociclib 50 mg/kg once daily, subcutaneous fulvestrant 5 mg/kg weekly, oral camizestrant 10 mg/kg once daily, capivasertib 130 mg/kg twice daily on a 4 days on, 3 days off dosing schedule. The triplet combination and the combination of camizestrant and capivasertib proved superior to the other doublet combinations and monotherapy arms.

FIG. 13. Combination efficacy of AZD9833/capivasertib doublet combination compared to fulvestrant/capivasertib doublet combination and monotherapy in an ST1799 model (ESR1 wt, PI3KCAm E542K). Patient-derived xenograft preclinical models were used to compare the indicated combinations and monotherapy. FIG. 13 shows tumor volume plots for ER+ breast cancer PDX in a 28-day efficacy study, displaying all treatment arms. Dosing: subcutaneous fulvestrant 5 mg/kg weekly, oral camizestrant 10 mg/kg once daily, capivasertib 130 mg/kg twice daily on a 4 days on, 3 days off dosing schedule. Monotherapy with fulvestrant and camizestrant delivered similar inhibition of tumor growth in this model. The combination of camizestrant and capivasertib was superior to the combination of fulvestrant and capivasertib and the monotherapy arms of the study.

FIG. 14. Combination efficacy of AZD9833/capivasertib doublet combination compared to fulvestrant/capivasertib doublet combination and monotherapy in an in an ST3632 model (ESR1 wt, AKT1m E17K). Patient-derived xenograft preclinical models were used to compare the indicated combinations and monotherapy. FIG. 14 shows tumor volume plots for ER+ breast cancer PDX in a 28-day efficacy study, displaying all treatment arms. Dosing: subcutaneous fulvestrant 5 mg/kg once weekly, oral camizestrant 10 mg/kg once daily, capivasertib 130 mg/kg twice daily on a 4 days on, 3 days off dosing schedule. The combination of camizestrant and capivasertib was superior to the combination of fulvestrant and capivasertib and the monotherapy arms of the study.

FIG. 15. Combination efficacy of AZD9833/capivasertib doublet combination compared to fulvestrant/capivasertib doublet combination and monotherapy in an ST941 model (ESR1m Y537S). Patient-derived xenograft preclinical models were used to compare the indicated combinations and monotherapy. FIG. 15 shows tumor volume plots for ER+ breast cancer PDX in a 28-day efficacy study, displaying all treatment arms. Dosing: subcutaneous fulvestrant 5 mg/kg once weekly, oral camizestrant 10 mg/kg once daily, capivasertib 130 mg/kg twice daily on a 4 days on, 3 days off dosing schedule. The combination of camizestrant and capivasertib was superior to the combination of fulvestrant and capivasertib.

FIG. 16. Combination efficacy of AZD9833/capivasertib doublet combination compared to fulvestrant/capivasertib doublet combination and monotherapy in a CTC174 model (altered PI3KCA/AKT or PTEN ER+: ESR1m D538G, PI3KCAm N345K). Xenograft preclinical models were used to compare the indicated combinations and monotherapy. FIG. 16 shows tumor volume plots for ER+ breast cancer PDX in a 28-day efficacy study, displaying all treatment arms. Dosing: subcutaneous fulvestrant 5 mg/kg once weekly, oral camizestrant 10 mg/kg once daily, capivasertib 130 mg/kg twice daily on a 4 days on, 3 days off dosing schedule. The combination of camizestrant and capivasertib was superior to the combination of fulvestrant and capivasertib and the monotherapy arms of the study.

### DETAILED DESCRIPTION

### Cancer Treatment

In embodiments the treatment of cancer is treatment of an animal cancer (for example a mammalian cancer such as a human cancer).

In embodiments the cancer is hormone-sensitive cancer (for example estrogen-sensitive cancer or androgen-sensitive cancer). “Estrogen or Androgen-sensitive” means that growth of the cancer is at least partially driven by the respective hormonal pathway, such that blocking the hormone attenuates growth and effects treatment.

In embodiments the cancer is breast cancer (for example early breast cancer, advanced breast cancer or metastatic breast cancer).

In embodiments the cancer is early breast cancer.

In embodiments the cancer is advanced breast cancer.

In embodiments the cancer is metastatic breast cancer.

In embodiments the cancer is hormone-sensitive breast cancer.

In embodiments the cancer is estrogen-sensitive breast cancer.

In embodiments the cancer is ovarian cancer.

In embodiments the cancer is estrogen-sensitive ovarian cancer.

In embodiments the cancer is endometrial cancer.

In embodiments the cancer is estrogen-sensitive endometrial cancer.

In embodiments the cancer is prostate cancer.

In embodiments the cancer is androgen-sensitive prostate cancer.

### Patient Selection and Diagnostic Methods

In embodiments the cancer is estrogen receptor positive (ER+) breast cancer.

“Estrogen receptor positive” cancer comprises tumours having the estrogen receptor (e.g. in at least 1%, at least 10%, at least 20%, or at least 50% of tumour cells) and are able to metabolise estrogen to grow. ER+ status can be determined by methods known in the art, for example by immunohistochemistry (IHC) tests.

In embodiments the cancer is estrogen receptor positive breast cancer.

In embodiments the cancer is breast cancer comprising only wild-type estrogen receptors. Such cancers contain no mutant estrogen receptors, only the receptors found in their normal state.

In embodiments the cancer is breast cancer comprises mutant estrogen receptors. Mutant estrogen receptors are synthesised by cancers harbouring mutations in their genetic structure (for example in the ESR1 gene). Mutations in the estrogen receptor can be determined by methods known in the art, for example by next generation sequencing.

In embodiments the cancer comprises no ESR1 mutations.

In embodiments the cancer comprises no ESR1 fusions.

In embodiments the cancer comprises no ESR1 mutations or fusions.

In embodiments the cancer comprises a mutation in ESR1 which is selected from an E380Q mutation, a Y537S mutation, and a D538G mutation.

In embodiments the cancer comprises an ESR1-CCDC170 fusion.

In embodiments the cancer comprises a mutation in ESR1 which is selected from an E380Q mutation, a Y537S mutation, and a D538G mutation and/or an ESR1-CCDC170 fusion.

In embodiments the cancer comprises a mutation in ESR1 which is selected from an E380Q mutation, a Y537S mutation, and a D538G mutation, and/or an ESR1-CCDC170 fusion.

In embodiments the cancer is PTEN deficient (e.g. comprises a cancerous cell (e.g. a population of cancerous cells, such as the majority of cancerous cells) with a reduction in the normal amount [e.g. compared to a non-cancerous cell of the same patient] or function of the PTEN tumour suppression protein). PTEN status can be determined by methods known in the art.

In embodiments the cancer comprises an AKT1 mutation (for example a gain of function mutation, or a deletion, substitution or insertion mutation, such as an E17K mutation). AKT1 mutation status can be determined by methods known in the art.

In embodiments the cancer comprises a PI3KCA mutation (for example a gain of function mutation, or a deletion, substitution or insertion mutation, such as a PI3KCA^{E542K}, PI3KCA^{E545K}, PI3KCA^{Q546R}, PI3KC^{A1047L} or PI3KCA^{H1047R} mutation). PI3KCA mutation status can be determined by methods known in the art.

In embodiments the PI3KCA mutation is selected from R88Q, C420R, E542K, E545A, E545D, E545Q, E545K, E545G, Q546E, Q546K, Q546R, Q546P, M1043V, M1043I, N345K, H1047Y, H1047R, H1047L and G1049R.

In embodiments the PI3KCA mutation is selected from R88Q, E542K, E545K and N354K.

In embodiments the PI3KCA mutation is selected from E545K and N345K.

In one embodiment there is provided a SERD for use in the treatment of cancer, where the SERD is administered in combination with an AKT inhibitor and the cancer is PTEN deficient, comprises an AKT1 mutation (for example an E17K AKT1 mutation) and/or comprises a PI3KCA mutation (for example a PI3KCA mutation selected from an E545K mutation and an N345K mutation).

In one embodiment there is provided a SERD for use in the treatment of cancer, where the SERD is administered in combination with an AKT inhibitor and the cancer is PTEN deficient, comprises an AKT1 mutation and comprises a PI3KCA mutation.

In one embodiment there is provided a SERD for use in the treatment of cancer, where the SERD is administered in combination with an AKT inhibitor and the cancer is PTEN deficient, comprises an AKT1 mutation or comprises a PI3KCA mutation.

In one embodiment there is provided a SERD for use in the treatment of cancer, where the SERD is administered in combination with an AKT inhibitor and the cancer is PTEN deficient.

In one embodiment there is provided a SERD for use in the treatment of cancer, where the SERD is administered in combination with an AKT inhibitor and the cancer comprises an AKT1 mutation and/or comprises a PI3KCA mutation.

In one embodiment there is provided a SERD for use in the treatment of cancer, where the SERD is administered in combination with an AKT inhibitor and the cancer comprises an AKT1 mutation and a PI3KCA mutation.

In one embodiment there is provided a SERD for use in the treatment of cancer, where the SERD is administered in combination with an AKT inhibitor and the cancer comprises an AKT1 mutation or a PI3KCA mutation.

In embodiments the cancer is estrogen receptor positive (ER+) breast cancer comprising a mutation in ESR1 (for example a mutation in ESR1 which is selected from an E380Q mutation, a Y537S mutation, and a D538G mutation); and/or an ESR1-CCDC170 fusion; and is PTEN deficient, comprises an AKT1 mutation (for example an E17K mutation) and/or comprises a PI3KCA mutation (for example a PI3KCA mutation selected from an E542K and an N345K mutation).

In embodiments the cancer is estrogen receptor positive (ER+) breast cancer comprising no ESR1 mutations or fusions and an E542K PI3KCA mutation.

In embodiments the cancer is estrogen receptor positive (ER+) breast cancer comprising no ESR1 mutations or fusions and an E17K AKT1 mutation.

In embodiments the cancer is estrogen receptor positive (ER+) breast cancer comprising a Y537S ESR1 mutation.

In embodiments the cancer is estrogen receptor positive (ER+) breast cancer which is PTEN deficient and comprises an R88Q PI3KCA mutation.

In embodiments the cancer is estrogen receptor positive (ER+) breast cancer comprising an E380Q ESR1 mutation and an N345K PI3KCA mutation.

In embodiments the cancer is estrogen receptor positive (ER+) breast cancer comprising a D538G ESR1 mutation and an E545K PI3KCA mutation.

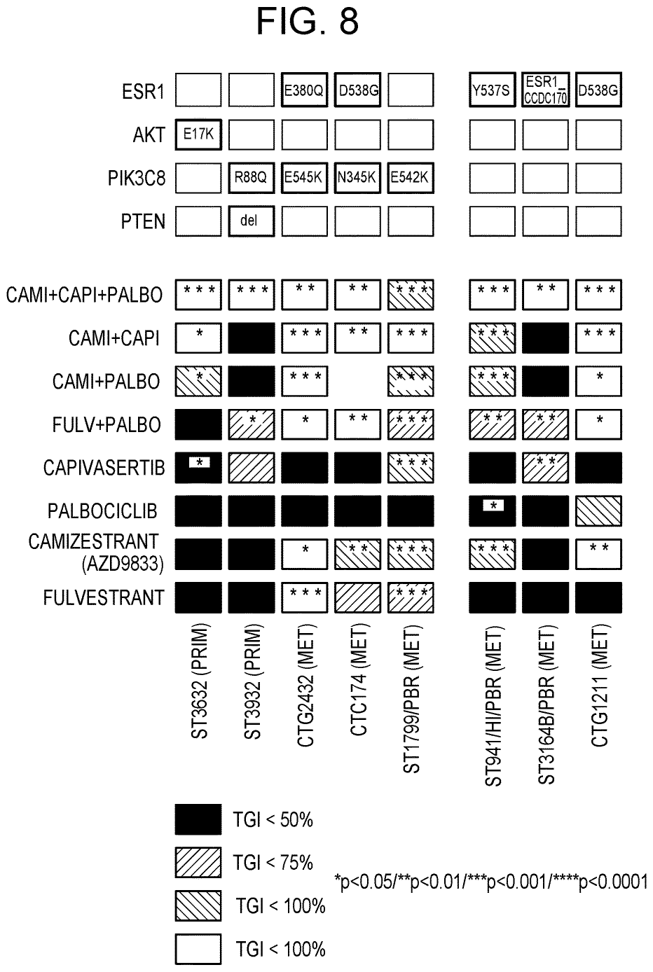

In embodiments the cancer is characterised by any of the biomarker profiles (for example genetic marker profiles) mentioned in the experimental section (e.g. the biomarkers associated with the cell lines listed in Table 2 and corresponding parts of the Figures and description [such as for example FIG. 8], alone or in combination).

In embodiments the cancer comprises a PIK3CA mutation and is characterised by ATM deletion, BCL2 deletion and/or MCL1 amplification.

In embodiments the cancer overexpresses Cdc6, Cyclin D1 and/or Cyclin E.

In embodiments the cancer overexpresses Cdc6 and/or is characterised by Rb loss.

In embodiments the cancer overexpresses CDK6 and/or CCNE1.

In embodiments the treatment of cancer is in a post-menopausal woman or a pre-menopausal woman.

A woman is an adult human female of the sex designed to produce large gametes (eggs).

In embodiments the treatment of cancer is in a post-menopausal woman.

In embodiments the treatment of cancer is in a pre-menopausal woman.

In embodiments the cancer has previously received treatment with a selective estrogen receptor degrader, a selective estrogen receptor modulator or an aromatase inhibitor.

In embodiments the human patient's cancer has reached the stage of maximal response (minimal residual disease) during or after treatment with a selective estrogen receptor degrader, a selective estrogen receptor modulator or an aromatase inhibitor.

In embodiments the cancer is resistant to treatment with a selective estrogen receptor degrader, a selective estrogen receptor modulator or an aromatase inhibitor.

In embodiments the cancer has progressed during or after previous treatment with a selective estrogen receptor degrader, a selective estrogen receptor modulator and/or an aromatase inhibitor. When a cancer's growth has “progressed”, its growth is no longer suitably controlled by the therapy in question.

In embodiments the human patient's cancer has reached the stage of maximal response (minimal residual disease) during or after treatment with fulvestrant or a pharmaceutical salt thereof.

In embodiments the cancer is resistant to treatment with fulvestrant or a pharmaceutical salt thereof.

In embodiments the cancer has progressed during or after previous treatment with fulvestrant or a pharmaceutical salt thereof.

In embodiments the cancer has previously received treatment with a CDK4/6 inhibitor.

In embodiments the cancer has reached the stage of maximal response (minimal residual disease) during or after treatment with a CDK4/6 inhibitor.

In embodiments the cancer is resistant to treatment with a CDK4/6 inhibitor.

In embodiments the human patient's cancer has reached the stage of maximal response (minimal residual disease) during or after treatment with palbociclib or a pharmaceutical salt thereof.

In embodiments the cancer is resistant to treatment with palbociclib or a pharmaceutical salt thereof.

In embodiments the cancer has progressed during or after previous treatment with palbociclib or a pharmaceutical salt thereof.

In embodiments the cancer is CCNE1 amplified (i.e. expresses greater than the normal amount of CCNE1 compared to a normal, healthy cell of the same type), RB1 deficient (i.e. expresses less than the normal amount of RB1compared to a normal, healthy cell of the same type), overexpresses CDC6 (i.e. expresses greater than the normal amount of CDC6 compared to a normal, healthy cell of the same type) and/or overexpresses CDK6 (i.e. expresses greater than the normal amount of CDK6 compared to a normal, healthy cell of the same type).

In embodiments the cancer is CCNE1 amplified and/or RB1 deficient.

In embodiments the cancer is resistant to treatment with a CDK4/6 inhibitor and is CCNE1 amplified, RB1 deficient, overexpresses CDC6 and/or overexpresses CDK6.

In embodiments the cancer is resistant to treatment with a CDK4/6 inhibitor and is CCNE1 amplified or RB1 deficient.

In embodiments the cancer has progressed during or after previous treatment with a CDK4/6 inhibitor. In embodiments the cancer has never had previous treatment with a CDK4/6 inhibitor.

In embodiments the cancer has the characteristics (for example biomarker characteristics) of any of the cell lines used in the experimental section (for example, the cell lines and biomarker characteristics shown in Table 2).

### Selective Estrogen Degraders

“Selective estrogen degraders” (SERDs) bind to the estrogen receptor causing it to be degraded and therefore downregulated.

In embodiments the selective estrogen degrader is a next generation selective estrogen degrader (“ngSERD”, for example giredestrant or a pharmaceutically acceptable salt thereof, elacestrant or a pharmaceutically acceptable salt thereof, imlunestrant or a pharmaceutically acceptable salt thereof or camizestrant or a pharmaceutically acceptable salt thereof).

In embodiments the selective estrogen receptor degrader is selected from fulvestrant or a pharmaceutically acceptable salt thereof, giredestrant or a pharmaceutically acceptable salt thereof, elacestrant or a pharmaceutically acceptable salt thereof, imlunestrant or a pharmaceutically acceptable salt thereof and camizestrant or a pharmaceutically acceptable salt thereof.

In embodiments the selective estrogen receptor degrader is selected from fulvestrant or a pharmaceutically acceptable salt thereof, giredestrant or a pharmaceutically acceptable salt thereof and elacestrant or a pharmaceutically acceptable salt thereof.

In embodiments the selective estrogen receptor degrader is selected from giredestrant or a pharmaceutically acceptable salt thereof, elacestrant or a pharmaceutically acceptable salt thereof, imlunestrant or a pharmaceutically acceptable salt thereof and camizestrant or a pharmaceutically acceptable salt thereof.

In embodiments the selective estrogen receptor degrader is selected from giredestrant or a pharmaceutically acceptable salt thereof and elacestrant or a pharmaceutically acceptable salt thereof.

In embodiments the selective estrogen receptor degrader is camizestrant or a pharmaceutically acceptable salt thereof.

In embodiments the selective estrogen receptor degrader is fulvestrant or a pharmaceutically acceptable salt thereof.

In embodiments the selective estrogen receptor degrader is giredestrant or a pharmaceutically acceptable salt thereof.

In embodiments the selective estrogen receptor degrader is imlunestrant or a pharmaceutically acceptable salt thereof.

In embodiments the selective estrogen receptor degrader is camizestrant or a pharmaceutically acceptable salt thereof.

In embodiments the selective estrogen receptor degrader is a PROTAC (proteolysis targeting chimera).

In embodiments the selective estrogen receptor degrader is ARV-471 or a pharmaceutically acceptable salt thereof.

Camizestrant (AZD9833) has the following chemical structure:

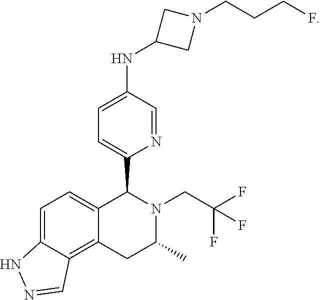 [SMILES: C[C@@H]1Cc2c(ccc3[nH]ncc23)[C@@H](c2ccc(NC3CN(CCCF)C3)cn2)N1CC(F)(F)F]

The free base of camizestrant is known by the chemical name N-(1-(3-fluoropropyl) azetidin-3-yl)-6-((6S,8R)-8-methyl-7-(2,2,2-trifluoroethyl)-6,7,8,9-tetrahydro-3H-pyrazolo[4,3-f]isoquinolin-6-yl)pyridin-3-amine. Camizestrant is disclosed in WO2018077630A1.

Imlunestrant (LY-3484356) has the following chemical structure:

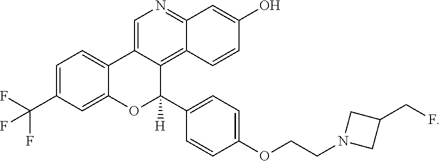 [SMILES: Oc1ccc2c3c(cnc2c1)-c1ccc(C(F)(F)F)cc1O[C@@H]3c1ccc(OCCN2CC(CF)C2)cc1]

The free base of imlunestrant is known by the chemical name (5R)-5-[4-[2-[3-(fluoromethyl) azetidin-1-yl]ethoxy]phenyl]-8-(trifluoromethyl)-5H-chromeno[4,3-c]quinolin-2-ol. Imlunestrant is disclosed in WO2020014435.

Giredestrant (GDC-9545) has the following chemical structure:

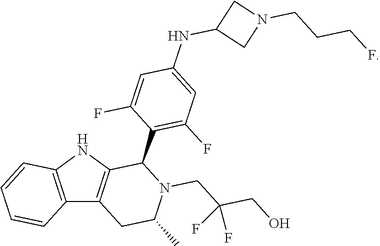 [SMILES: C[C@@H]1Cc2c([nH]c3ccccc23)[C@@H](c2c(F)cc(NC3CN(CCCF)C3)cc2F)N1CC(F)(F)CO]

The free base of giredestrant is known by the chemical name 3-[(1R,3R)-1-[2,6-difluoro-4-[[1-(3-fluoropropyl) azetidin-3-yl]amino]phenyl]-3-methyl-1,3,4,9-tetrahydropyrido[3,4-b]indol-2-yl]-2,2-difluoropropan-1-ol. Giredestrant is disclosed in WO2016097072A1.

ARV-471 has the following chemical structure:

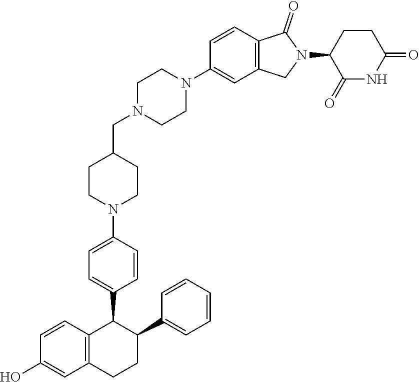 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

ARV-471 is disclosed in WO2018102725.

### AKT Inhibitors

In one embodiment there is provided a SERD for use in the treatment of cancer, where the SERD is administered in combination with an AKT inhibitor.

In one embodiment there is provided an AKT inhibitor for use in the treatment of cancer, where the AKT inhibitor is administered in combination with a SERD.

In embodiments the AKT inhibitor is any molecule or compound which binds to and inhibits the activity of one or more AKT isoforms (for example having a pIC₅₀ of >4.5, >5, >6, >7, >8 or >9 vs. the isoform in question).

In embodiments the AKT inhibitor is a proteolysis targeting chimera (PROTAC).

In embodiments the AKT inhibitor is selected from miransertib (ARQ-092) or a pharmaceutically acceptable salt thereof, BAY1125976 or a pharmaceutically acceptable salt thereof, borussertib or a pharmaceutically acceptable salt thereof, AT7867 or a pharmaceutically acceptable salt thereof, CCT128930 or a pharmaceutically acceptable salt thereof, A-674563 or a pharmaceutically acceptable salt thereof, PHT-427 or a pharmaceutically acceptable salt thereof, Akti-1/2 or a pharmaceutically acceptable salt thereof, AT13148 or a pharmaceutically acceptable salt thereof, SC79 or a pharmaceutically acceptable salt thereof, capivasertib or a pharmaceutically acceptable salt thereof, miltefosine or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, afuresertib (GSK2110183) or a pharmaceutically acceptable salt thereof, uprosertib (GSK2141795) or a pharmaceutically acceptable salt thereof, XL-418 or a pharmaceutically acceptable salt thereof and ipatasertib (GDC-0068) or a pharmaceutically acceptable salt thereof.

In embodiments the AKT inhibitor is selected from capivasertib or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, uprosertib (GSK2141795) or a pharmaceutically acceptable salt thereof, XL-418 or a pharmaceutically acceptable salt thereof and ipatasertib or a pharmaceutically acceptable salt thereof.

In embodiments the AKT inhibitor is selected from capivasertib or a pharmaceutically acceptable salt thereof, perifosine or a pharmaceutically acceptable salt thereof, MK-2206 or a pharmaceutically acceptable salt thereof, GSK690693 or a pharmaceutically acceptable salt thereof, afuresertib (GSK2110183) or a pharmaceutically acceptable salt thereof, uprosertib (GSK2141795) or a pharmaceutically acceptable salt thereof and ipatasertib (GDC-0068) or a pharmaceutically acceptable salt thereof.

In embodiments the AKT inhibitor is capivasertib or a pharmaceutically acceptable salt thereof.

Capivasertib has the following chemical structure:

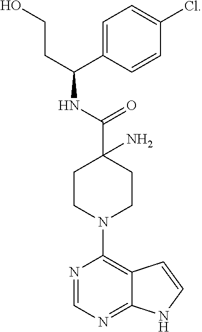 [SMILES: NC1(C(=O)N[C@@H](CCO)c2ccc(Cl)cc2)CCN(c2ncnc3[nH]ccc23)CC1]

The free base of capivasertib is known by the chemical name(S)-4-amino-N-(1-(4-chlorophenyl)-3-hydroxypropyl)-1-(7H-pyrrolo[2,3-d]pyrimidin-4-yl) piperidine-4-carboxamide). Capivasertib is disclosed in WO2009/047563, which discloses capivasertib (in Example 9) and describes its synthesis.

Perifosine has the following chemical structure:

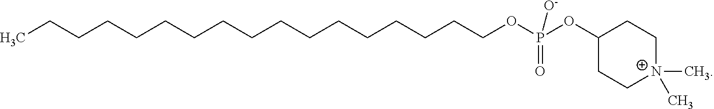 [SMILES: CCCCCCCCCCCCCCCCCOP(=O)([O-])OC1CC[N+](C)(C)CC1]

Perifosine is known by the chemical name 1,1-Dimethylpiperidinium-4-yl octadecyl phosphate.

Perifosine is disclosed in U.S. Pat. No. 8,383,607.

MK-2206 has the following chemical structure:

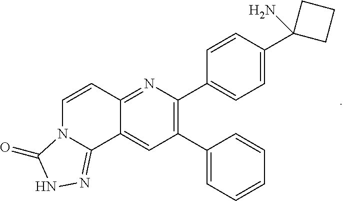 [SMILES: NC1(c2ccc(-c3nc4ccn5c(=O)[nH]nc5c4cc3-c3ccccc3)cc2)CCC1]

The free base of MK-2206 is known by the chemical name 8-[4-(1-Aminocyclobutyl)phenyl]-9-phenyl[1,2,4]triazolo[3,4-f][1,6]naphthyridin-3 (2H)-one. MK-2206 is disclosed in WO2008070016.

GSK690693 has the following chemical structure:

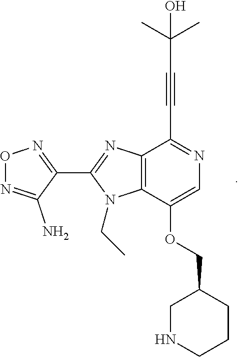 [SMILES: CCn1c(-c2nonc2N)nc2c(C#CC(C)(C)O)ncc(OC[C@H]3CCCNC3)c21]

The free base of GSK690693 is known by the chemical name 4-(2-(4-Amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol. GSK690693 is disclosed in WO2007058850.

Afuresertib (GSK2110183) has the following chemical structure:

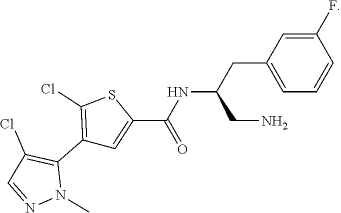 [SMILES: Cn1ncc(Cl)c1-c1cc(C(=O)N[C@H](CN)Cc2cccc(F)c2)sc1Cl]

The free base of afuresertib is known by the chemical name N-[(1S)-2-amino-1-[(3-fluorophenyl)methyl]ethyl]-5-chloro-4-(4-chloro-1-methyl-1H-pyrazol-5-yl)-2-thiophenecarboxamide.

Afuresertib is disclosed in WO2008098104.

Uprosertib (GSK2141795) has the following chemical structure:

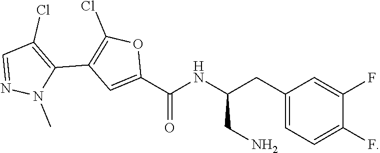 [SMILES: Cn1ncc(Cl)c1-c1cc(C(=O)N[C@H](CN)Cc2ccc(F)c(F)c2)oc1Cl]

The free base of uprosertib is known by the chemical name N-[(1S)-2-amino-1-[(3,4-difluorophenyl)methyl]ethyl]-5-chloro-4-(4-chloro-1-methyl-1H-pyrazol-5-yl)-2-furancarboxamide.

Uprosertib is disclosed in WO2008098104.

Ipatasertib has the following chemical structure:

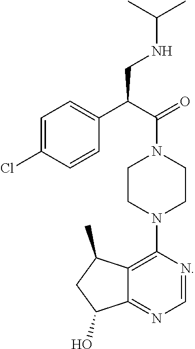 [SMILES: CC(C)NC[C@@H](C(=O)N1CCN(c2ncnc3c2[C@H](C)C[C@H]3O)CC1)c1ccc(Cl)cc1]

The free base of ipatasertib is known by the chemical name 2-(4-chlorophenyl)-1-(4-((5R,7R)-7-hydroxy-5-methyl-6,7-dihydro-5H-cyclopenta[d]pyrimidin-4-yl) piperazin-1-yl)-3-(isopropylamino) propan-1-one.

Ipatasertib is disclosed in WO2008006040.

mTOR Inhibitors

In one embodiment there is provided a SERD for use in the treatment of cancer, where the SERD is administered in combination with an mTOR inhibitor.

In one embodiment there is provided an mTOR inhibitor for use in the treatment of cancer, where the mTOR inhibitor is administered in combination with a SERD.

In embodiments the mTOR inhibitor is any molecule or compound which binds to and inhibits the activity of mTOR (for example having a pIC₅₀ of >4.5, >5, >6, >7, >8 or >9 vs. mTOR). In embodiments the mTOR inhibitor is an mTORC1 inhibitor.

In embodiments the mTOR inhibitor is an mTORC1 selective inhibitor. An mTORC1 selective inhibitor has greater activity (for example >10-fold, >100-fold or >1000-fold activity) against mTORC1 than against any other mTOR complex.

In embodiments the mTOR inhibitor is selected from everolimus (e.g. Afinitor®) or a pharmaceutically acceptable salt thereof and temsirolimus (e.g. Torisel®) or a pharmaceutically acceptable salt thereof. In embodiments the mTOR inhibitor is everolimus or a pharmaceutically acceptable salt thereof.

In embodiments the mTOR inhibitor is temsirolimus or a pharmaceutically acceptable salt thereof.

### CDK4/6 Inhibitors

In one embodiment there is provided a SERD for use in the treatment of cancer, where the SERD is administered in combination with a CDK4/6 inhibitor.

In one embodiment there is provided a CDK4/6 inhibitor for use in the treatment of cancer, where the CDK4/6 inhibitor is administered in combination with a SERD.

In one embodiment there is provided a ngSERD (for example giredestrant or a pharmaceutically acceptable salt thereof, elacestrant or a pharmaceutically acceptable salt thereof, imlunestrant or a pharmaceutically acceptable salt thereof or camizestrant or a pharmaceutically acceptable salt thereof) for use in the treatment of cancer, where the ngSERD is administered in combination with a CDK4/6 inhibitor.

In one embodiment there is provided a CDK4/6 inhibitor for use in the treatment of cancer, where the CDK4/6 inhibitor is administered in combination with a ngSERD.

In embodiments the CDK4/6 inhibitor is any molecule or compound which binds to and inhibits the activity of CDK4 and CDK6 (for example having a pIC₅₀ of >4.5, >5, >6, >7, >8 or >9 vs. CDK4 and CDK6). In embodiments the CDK4/6 inhibitor is selected from palbociclib (e.g. Ibrance®) or a pharmaceutically acceptable salt thereof, ribociclib (e.g. Kisqali®) or a pharmaceutically acceptable salt thereof and abemaciclib (e.g. Verzenios®) or a pharmaceutically acceptable salt thereof.

In embodiments the CDK4/6 inhibitor is palbociclib or a pharmaceutically acceptable salt thereof.

In embodiments the CDK4/6 inhibitor is ribociclib or a pharmaceutically acceptable salt thereof.

In embodiments the CDK4/6 inhibitor is abemaciclib or a pharmaceutically acceptable salt thereof.

### Other Endocrine Therapies

“Selective estrogen modulators” (SERMs) are compounds that agonise or antagonise the estrogen receptor, often differently depending on which tissue they act. In embodiments the selective estrogen modulator has an anti-estrogenic effect on cancer. In embodiments the selective estrogen receptor modulator is selected from tamoxifen (e.g. Nolvadex®) or a pharmaceutically acceptable salt thereof, toremifene (e.g. Fareston®) or a pharmaceutically acceptable salt thereof and raloxifene (e.g. Evista®) or a pharmaceutically acceptable salt thereof.

In embodiments the SERM is tamoxifen or a pharmaceutically acceptable salt thereof.

In embodiments the SERM is toremifene or a pharmaceutically acceptable salt thereof.

In embodiments the SERM is raloxifene or a pharmaceutically acceptable salt thereof.

“Aromatase inhibitors” are compounds that block the biosynthesis of estrogen. In embodiments the aromatase inhibitor is selected from anastrozole (e.g. Arimidex®) or a pharmaceutically acceptable salt thereof, letrozole (e.g. Femara®) or a pharmaceutically acceptable salt thereof and exemestane (e.g. Aromasin®) or a pharmaceutically acceptable salt thereof.

In embodiments the aromatase inhibitor is anastrozole or a pharmaceutically acceptable salt thereof.

In embodiments the aromatase inhibitor is letrozole or a pharmaceutically acceptable salt thereof.

In embodiments the aromatase inhibitor is exemestane or a pharmaceutically acceptable salt thereof.

### Triplet Combinations

In one embodiment there is provided a SERD for use in the treatment of cancer, where the SERD is administered in combination with an AKT inhibitor or an mTOR inhibitor and a CDK4/6 inhibitor.

In one embodiment there is provided an AKT inhibitor or an mTOR inhibitor for use in the treatment of cancer, where the AKT inhibitor or an mTOR inhibitor is administered in combination with a SERD and a CDK4/6 inhibitor.

In one embodiment there is provided an AKT inhibitor for use in the treatment of cancer, where the AKT inhibitor is administered in combination with a SERD and a CDK4/6 inhibitor.

In one embodiment there is provided an mTOR inhibitor for use in the treatment of cancer, where the mTOR inhibitor is administered in combination with a SERD and a CDK4/6 inhibitor.

In one embodiment there is provided a CDK4/6 inhibitor for use in the treatment of cancer, where the CDK4/6 inhibitor is administered in combination with a SERD and an AKT inhibitor or an mTOR inhibitor.

In embodiments administration of the SERD and each inhibitor is separate, sequential, or simultaneous.

In embodiments administration of the SERD and each inhibitor is separate.

In embodiments administration of the SERD and each inhibitor is sequential.

In embodiments administration of the SERD and each inhibitor is simultaneous.

### Specific Combinations

In one embodiment there is provided an AKT inhibitor or an mTOR inhibitor for use in the treatment of cancer, where the AKT inhibitor or an mTOR inhibitor is administered in combination with a SERD and optionally a CDK4/6 inhibitor.

In one embodiment there is provided a SERD for use in the treatment of cancer, where the SERD is administered in combination with an AKT inhibitor selected from miransertib or a pharmaceutically acceptable salt thereof, BAY1125976 or a pharmaceutically acceptable salt thereof, borussertib or a pharmaceutically acceptable salt thereof, AT7867 or a pharmaceutically acceptable salt thereof, CCT128930 or a pharmaceutically acceptable salt thereof, A-674563 or a pharmaceutically acceptable salt thereof, PHT-427 or a pharmaceutically acceptable salt thereof, Akti-1/2 or a pharmaceutically acceptable salt thereof, AT13148 or a pharmaceutically acceptable salt thereof, SC79 or a pharmaceutically acceptable salt thereof, capivasertib or a pharmaceutically acceptable salt thereof, miltefosine or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, afuresertib or a pharmaceutically acceptable salt thereof, uprosertib or a pharmaceutically acceptable salt thereof, XL-418 or a pharmaceutically acceptable salt thereof and ipatasertib or a pharmaceutically acceptable salt thereof or an mTOR inhibitor selected from everolimus or a pharmaceutically acceptable salt thereof and temsirolimus or a pharmaceutically acceptable salt thereof, and/or a CDK4/6 inhibitor selected from palbociclib or a pharmaceutically acceptable salt thereof, ribociclib or a pharmaceutically acceptable salt thereof and abemaciclib or a pharmaceutically acceptable salt thereof.

As can be seen from the data presented herein, combination therapy with camizestrant and capivasertib delivers superior activity in ESR1 wt and ESR1m patient derived xenograft (PDX) models relative to therapy with fulvestrant and capivasertib. It appears that camizestrant and capivasertib act in a synergistic manner. This surprisingly advantageous combination generally delivers greater response across a range of ESR1 wt and ESR1m PDX models than is observed with monotherapy using either agent.

Furthermore, the combination of camizestrant and capivasertib delivered deeper response than that obtained with the optimal dose of fulvestrant in combination with capivasertib. The clinical potential of combination therapy with camizestrant and capivasertib is thus revealed for the first time, a significant finding given the results of the Phase 3 CAPitello-291clinical trial in which the combination of capivasertib and fulvestrant and demonstrated a statistically significant and clinically meaningful improvement in progression-free survival (PFS) versus placebo plus Faslodex in patients with hormone receptor (HR)-positive, HER2−low or negative, locally advanced or metastatic breast cancer, following recurrence or progression on, or after, endocrine therapy (with or without a CDK4/6 inhibitor)

Triplet combinations comprising camizestrant and capivasertib in combination with a CDK4/6 inhibitor are observed to deliver excellent, synergistic, activity across a wide range of PDX models for example those with clinically relevant mutations to ESR1, in the AKT/PI3K pathway or in PDX models in which CCNE1 amplified or that are RB1 deficient or that overexpress CDC6 and/or that overexpress CDK6. In embodiments there are provided camizestrant, or a pharmaceutically acceptable salt thereof, for use in the treatment of cancer, wherein camizestrant, or a pharmaceutically acceptable salt thereof, is administered in combination with capivasertib, or a pharmaceutically acceptable salt thereof, optionally wherein the use further comprises the administration of a CDK4/6 inhibitor. In such embodiments, the camizestrant or a pharmaceutically acceptable salt thereof may be administered at a dose of 75 mg or 150 mg once daily.

In embodiments there are provided capivasertib for use in the treatment of cancer, wherein capivasertib, or a pharmaceutically acceptable salt thereof, is administered in combination with camizestrant, or a pharmaceutically acceptable salt thereof, optionally wherein the use further comprises the administration of a CDK4/6 inhibitor. In such embodiments, the capivasertib or a pharmaceutically acceptable salt thereof may be administered under an intermittent dosing schedule at a dosage of 400 mg twice daily.

In embodiments there are provided camizestrant, or a pharmaceutically acceptable salt thereof, for use in the treatment of cancer, wherein camizestrant, or a pharmaceutically acceptable salt thereof, is administered in combination with capivasertib or a pharmaceutically acceptable salt thereof.

In embodiments there are provided capivasertib, or a pharmaceutically acceptable salt thereof, for use in the treatment of cancer, wherein capivasertib, or a pharmaceutically acceptable salt thereof, is administered in combination with camizestrant, or a pharmaceutically acceptable salt thereof.

In embodiments there are provided camizestrant, or a pharmaceutically acceptable salt thereof, for use in the treatment of cancer, wherein camizestrant, or a pharmaceutically acceptable salt thereof, is administered in combination with capivasertib, or a pharmaceutically acceptable salt thereof, and a CDK4/6 inhibitor. In embodiments there are provided capivasertib or a pharmaceutically acceptable salt thereof for use in the treatment of cancer, wherein capivasertib or a pharmaceutically acceptable salt thereof is administered in combination with camizestrant or a pharmaceutically acceptable salt thereof and a CDK4/6 inhibitor.

In embodiments there are provided camizestrant, or a pharmaceutically acceptable salt thereof, for use in the treatment of cancer, wherein camizestrant, or a pharmaceutically acceptable salt thereof, is administered in combination with capivasertib, or a pharmaceutically acceptable salt thereof, optionally wherein the use further comprises the administration of a CDK4/6 inhibitor, and wherein the cancer is estrogen receptor positive (ER+) breast cancer comprising no ESR1 mutations or fusions.

In embodiments there are provided camizestrant, or a pharmaceutically acceptable salt thereof, for use in the treatment of cancer, wherein camizestrant, or a pharmaceutically acceptable salt thereof, is administered in combination with capivasertib, or a pharmaceutically acceptable salt thereof, optionally wherein the use further comprises the administration of a CDK4/6 inhibitor, and wherein the cancer is estrogen receptor positive (ER+) breast cancer comprising a mutation in ESR1, optionally wherein the mutation in ESR1 is selected from an E380Q mutation, a Y537S mutation, and a D538G mutation, and/or an ESR1-CCDC170 fusion.

In embodiments there are provided camizestrant, or a pharmaceutically acceptable salt thereof, for use in the treatment of cancer, wherein camizestrant, or a pharmaceutically acceptable salt thereof, is administered in combination with capivasertib, or a pharmaceutically acceptable salt thereof, optionally wherein the use further comprises the administration of a CDK4/6 inhibitor, and wherein the cancer is estrogen receptor positive (ER+) breast cancer comprising a mutation in ESR1, optionally wherein the mutation in ESR1 is selected from a Y537S mutation and a D538G mutation.

In embodiments there are provided camizestrant, or a pharmaceutically acceptable salt thereof, for use in the treatment of cancer, wherein camizestrant or a pharmaceutically acceptable salt thereof is administered in combination with capivasertib, or a pharmaceutically acceptable salt thereof, optionally wherein the use further comprises the administration of a CDK4/6 inhibitor, and wherein the cancer is PTEN deficient, comprises an AKT1 mutation (for example an E17K mutation) and/or comprises a PI3KCA mutation (for example a PI3KCA mutation selected from an E542K and an N345K mutation).

In embodiments there are provided camizestrant, or a pharmaceutically acceptable salt thereof, for use in the treatment of cancer, wherein camizestrant or a pharmaceutically acceptable salt thereof is administered in combination with capivasertib, or a pharmaceutically acceptable salt thereof, optionally wherein the use further comprises the administration of a CDK4/6 inhibitor, wherein the cancer is estrogen receptor positive (ER+) breast cancer comprising a mutation in ESR1, optionally a mutation in ESR1 which is selected from an E380Q mutation, a Y537S mutation, and a D538G mutation, and/or an ESR1-CCDC170 fusion; and wherein the cancer is PTEN deficient, comprises an AKT1 mutation (for example an E17K mutation) and/or comprises a PI3KCA mutation (for example a PI3KCA mutation selected from an E542K and an N345K mutation).

In embodiments there are provided camizestrant, or a pharmaceutically acceptable salt thereof, for use in the treatment of cancer, wherein camizestrant, or a pharmaceutically acceptable salt thereof, is administered in combination with capivasertib, or a pharmaceutically acceptable salt thereof, optionally wherein the use further comprises the administration of a CDK4/6 inhibitor, wherein the cancer is estrogen receptor positive (ER+) breast cancer comprising a mutation in ESR1, optionally wherein the mutation in ESR1 is selected from a Y537S mutation and a D538G mutation, and wherein the cancer comprises an E17K AKT1 mutation and/or a PI3KCA mutation selected from an E542K and an N345K mutation.

In embodiments there are provided capivasertib, or a pharmaceutically acceptable salt thereof, for use in the treatment of cancer, wherein capivasertib, or a pharmaceutically acceptable salt thereof, is administered in combination with camizestrant, or a pharmaceutically acceptable salt thereof, optionally wherein the use further comprises the administration of a CDK4/6 inhibitor, and wherein the cancer is estrogen receptor positive (ER+) breast cancer comprising no ESR1 mutations or fusions.

In embodiments there are provided capivasertib, or a pharmaceutically acceptable salt thereof, for use in the treatment of cancer, wherein capivasertib, or a pharmaceutically acceptable salt thereof, is administered in combination with camizestrant, or a pharmaceutically acceptable salt thereof, optionally wherein the use further comprises the administration of a CDK4/6 inhibitor, and wherein the cancer is estrogen receptor positive (ER+) breast cancer comprising a mutation in ESR1, optionally wherein the mutation in ESR1 is selected from an E380Q mutation, a Y537S mutation, and a D538G mutation, and/or an ESR1-CCDC170 fusion.

In embodiments there are provided capivasertib, or a pharmaceutically acceptable salt thereof, for use in the treatment of cancer, wherein capivasertib, or a pharmaceutically acceptable salt thereof, is administered in combination with camizestrant, or a pharmaceutically acceptable salt thereof, optionally wherein the use further comprises the administration of a CDK4/6 inhibitor, and wherein the cancer is estrogen receptor positive (ER+) breast cancer comprising a mutation in ESR1, optionally wherein the mutation in ESR1 is selected from a Y537S mutation and a D538G mutation.

In embodiments there are provided capivasertib, or a pharmaceutically acceptable salt thereof, for use in the treatment of cancer, wherein capivasertib, or a pharmaceutically acceptable salt thereof, is administered in combination with camizestrant, or a pharmaceutically acceptable salt thereof, optionally wherein the use further comprises the administration of a CDK4/6 inhibitor, and wherein the cancer is PTEN deficient, comprises an AKT1 mutation (for example an E17K mutation) and/or comprises a PI3KCA mutation (for example a PI3KCA mutation selected from an E542K and an N345K mutation).

In embodiments there are provided capivasertib, or a pharmaceutically acceptable salt thereof, for use in the treatment of cancer, wherein capivasertib or a pharmaceutically acceptable salt thereof is administered in combination with camizestrant, or a pharmaceutically acceptable salt thereof, optionally wherein the use further comprises the administration of a CDK4/6 inhibitor, wherein the cancer is estrogen receptor positive (ER+) breast cancer comprising a mutation in ESR1, optionally wherein the mutation in ESR1 is selected from an E380Q mutation, a Y537S mutation, and a D538G mutation, and/or an ESR1-CCDC170 fusion; and wherein the cancer is PTEN deficient, comprises an AKT1 mutation (for example an E17K mutation) and/or comprises a PI3KCA mutation (for example a PI3KCA mutation selected from an E542K and an N345K mutation).

In embodiments there are provided capivasertib, or a pharmaceutically acceptable salt thereof, for use in the treatment of cancer, wherein capivasertib, or a pharmaceutically acceptable salt thereof, is administered in combination with camizestrant or a pharmaceutically acceptable salt thereof, optionally wherein the use further comprises the administration of a CDK4/6 inhibitor, wherein the cancer is estrogen receptor positive (ER+) breast cancer comprising a mutation in ESR1 which is selected from a Y537S mutation and a D538G mutation; and wherein the cancer comprises an E17K AKT1 mutation and/or a PI3KCA mutation selected from an E542K and an N345K mutation.

In one embodiment there is provided a SERD for use in the treatment of cancer, where the SERD is administered in combination with an AKT inhibitor selected from miransertib or a pharmaceutically acceptable salt thereof, BAY1125976 or a pharmaceutically acceptable salt thereof, borussertib or a pharmaceutically acceptable salt thereof, AT7867 or a pharmaceutically acceptable salt thereof, CCT128930 or a pharmaceutically acceptable salt thereof, A-674563 or a pharmaceutically acceptable salt thereof, PHT-427 or a pharmaceutically acceptable salt thereof, Akti-1/2 or a pharmaceutically acceptable salt thereof, AT13148 or a pharmaceutically acceptable salt thereof, SC79 or a pharmaceutically acceptable salt thereof, capivasertib or a pharmaceutically acceptable salt thereof, miltefosine or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, afuresertib or a pharmaceutically acceptable salt thereof, uprosertib or a pharmaceutically acceptable salt thereof, XL-418 or a pharmaceutically acceptable salt thereof and ipatasertib or a pharmaceutically acceptable salt thereof and/or a CDK4/6 inhibitor selected from palbociclib or a pharmaceutically acceptable salt thereof, ribociclib or a pharmaceutically acceptable salt thereof and abemaciclib or a pharmaceutically acceptable salt thereof.

In one embodiment there is provided a SERD for use in the treatment of cancer, where the SERD is administered in combination with an mTOR inhibitor selected from everolimus or a pharmaceutically acceptable salt thereof and temsirolimus or a pharmaceutically acceptable salt thereof, and/or a CDK4/6 inhibitor selected from palbociclib or a pharmaceutically acceptable salt thereof, ribociclib or a pharmaceutically acceptable salt thereof and abemaciclib or a pharmaceutically acceptable salt thereof.

In one embodiment there is provided a SERD for use in the treatment of cancer, where the SERD is administered in combination with an AKT inhibitor selected from miransertib or a pharmaceutically acceptable salt thereof, BAY1125976 or a pharmaceutically acceptable salt thereof, borussertib or a pharmaceutically acceptable salt thereof, AT7867 or a pharmaceutically acceptable salt thereof, CCT128930 or a pharmaceutically acceptable salt thereof, A-674563 or a pharmaceutically acceptable salt thereof, PHT-427 or a pharmaceutically acceptable salt thereof, Akti-1/2 or a pharmaceutically acceptable salt thereof, AT13148 or a pharmaceutically acceptable salt thereof, SC79 or a pharmaceutically acceptable salt thereof, capivasertib or a pharmaceutically acceptable salt thereof, miltefosine or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, afuresertib or a pharmaceutically acceptable salt thereof, uprosertib or a pharmaceutically acceptable salt thereof, XL-418 or a pharmaceutically acceptable salt thereof and ipatasertib or a pharmaceutically acceptable salt thereof or an mTOR inhibitor selected from everolimus or a pharmaceutically acceptable salt thereof and temsirolimus or a pharmaceutically acceptable salt thereof, and a CDK4/6 inhibitor selected from palbociclib or a pharmaceutically acceptable salt thereof, ribociclib or a pharmaceutically acceptable salt thereof and abemaciclib or a pharmaceutically acceptable salt thereof.

In one embodiment there is provided a SERD for use in the treatment of cancer, where the SERD is administered in combination with an AKT inhibitor selected from miransertib or a pharmaceutically acceptable salt thereof, BAY1125976 or a pharmaceutically acceptable salt thereof, borussertib or a pharmaceutically acceptable salt thereof, AT7867 or a pharmaceutically acceptable salt thereof, CCT128930 or a pharmaceutically acceptable salt thereof, A-674563 or a pharmaceutically acceptable salt thereof, PHT-427 or a pharmaceutically acceptable salt thereof, Akti-1/2 or a pharmaceutically acceptable salt thereof, AT13148 or a pharmaceutically acceptable salt thereof, SC79 or a pharmaceutically acceptable salt thereof, capivasertib or a pharmaceutically acceptable salt thereof, miltefosine or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, afuresertib or a pharmaceutically acceptable salt thereof, uprosertib or a pharmaceutically acceptable salt thereof, XL-418 or a pharmaceutically acceptable salt thereof and ipatasertib or a pharmaceutically acceptable salt thereof and a CDK4/6 inhibitor selected from palbociclib or a pharmaceutically acceptable salt thereof, ribociclib or a pharmaceutically acceptable salt thereof and abemaciclib or a pharmaceutically acceptable salt thereof.

In one embodiment there is provided a SERD for use in the treatment of cancer, where the SERD is administered in combination with an mTOR inhibitor selected from everolimus or a pharmaceutically acceptable salt thereof and temsirolimus or a pharmaceutically acceptable salt thereof, and a CDK4/6 inhibitor selected from palbociclib or a pharmaceutically acceptable salt thereof, ribociclib or a pharmaceutically acceptable salt thereof and abemaciclib or a pharmaceutically acceptable salt thereof.

In one embodiment there is provided a SERD for use in the treatment of cancer, where the SERD is camizestrant or a pharmaceutically acceptable salt thereof which is administered in combination with abemaciclib, and the cancer is resistant to treatment with palbociclib.

### Pharmaceutical Compositions and Dosage Forms

In one embodiment there is provided a pharmaceutical composition comprising a SERD in combination with an AKT inhibitor or an mTOR inhibitor, and/or a CDK4/6 inhibitor, and a pharmaceutically acceptable excipient.

“Pharmaceutically acceptable excipients” include diluents, disintegrants or lubricants. In a further embodiment, the pharmaceutical composition comprises one or more pharmaceutical diluents (such as mannitol and microcrystalline cellulose), one or more pharmaceutical disintegrants (such as low-substituted hydroxypropyl cellulose) or one or more pharmaceutical lubricants (such as sodium stearyl fumarate).

In one embodiment there is provided a pharmaceutical composition comprising a SERD in combination with an AKT inhibitor or an mTOR inhibitor and a pharmaceutically acceptable excipient.

In one embodiment there is provided a pharmaceutical composition comprising a SERD in combination with a CDK4/6 inhibitor and a pharmaceutically acceptable excipient.

In one embodiment there is provided a pharmaceutical composition comprising a SERD in combination with an AKT inhibitor or an mTOR inhibitor, a CDK4/6 inhibitor, and a pharmaceutically acceptable excipient.

In embodiments the composition is an oral dosage form.

In embodiments the composition is in the form of a tablet or capsule.

In embodiments camizestrant or a pharmaceutically acceptable salt thereof is administered to the subject at a daily dosage of 75 mg or 150 mg.

In the combinations disclosed in this specification, capivasertib, or a pharmaceutically acceptable salt thereof, is generally administered to the subject at a daily dosage from about 100 mg to about 1600 mg.

In embodiments capivasertib, or a pharmaceutically acceptable salt thereof, is administered at a daily dosage from about 150 mg to about 1500 mg. In embodiments capivasertib, or a pharmaceutically acceptable salt thereof, is administered at a daily dosage from about 200 mg to about 1400 mg. In another embodiment, capivasertib, or a pharmaceutically acceptable salt thereof, is administered at a daily dosage from about 300 mg to about 1300 mg. In another embodiment, capivasertib, or a pharmaceutically acceptable salt thereof, is administered at a daily dosage from about 400 mg to about 1200 mg. In another embodiment, capivasertib, or a pharmaceutically acceptable salt thereof, is administered at a daily dosage from about 500 mg to about 1100 mg. In another embodiment, capivasertib, or a pharmaceutically acceptable salt thereof, is administered at a daily dosage from about 600 mg to about 1000 mg. In embodiments capivasertib, or a pharmaceutically acceptable salt thereof, is administered to the subject once daily (QD).

In embodiments capivasertib, or a pharmaceutically acceptable salt thereof, is administered at a dosage from about 100 mg to about 1000 mg once daily.

In another embodiment, capivasertib, or a pharmaceutically acceptable salt thereof, is administered at a dosage from about 150 mg to about 900 mg once daily. In another embodiment, capivasertib, or a pharmaceutically acceptable salt thereof, is administered at a dosage from about 200 mg to about 850 mg once daily. In another embodiment, capivasertib, or a pharmaceutically acceptable salt thereof, is administered at a dosage from about 250 mg to about 800 mg once daily. In another embodiment, capivasertib, or a pharmaceutically acceptable salt thereof, is administered at a dosage from about 300 mg to about 750 mg once daily.

In another embodiment, capivasertib, or a pharmaceutically acceptable salt thereof, is administered at a dosage from about 350 mg to about 700 mg once daily. In another embodiment, capivasertib, or a pharmaceutically acceptable salt thereof, is administered at a dosage from about 400 mg to about 650 mg once daily.

In embodiments capivasertib, or a pharmaceutically acceptable salt thereof, is administered to the subject twice daily (BID). In one embodiment, capivasertib, or a pharmaceutically acceptable salt thereof, is administered at a dosage from about 50 mg to about 900 mg twice daily. In another embodiment, capivasertib, or a pharmaceutically acceptable salt thereof, is administered at a dosage from about 100 mg to about 875 mg twice daily. In another embodiment, capivasertib, or a pharmaceutically acceptable salt thereof, is administered at a dosage from about 200 mg to about 850 mg twice daily. In another embodiment, capivasertib, or a pharmaceutically acceptable salt thereof, is administered at a dosage from about 250 mg to about 825 mg twice daily. In another embodiment, capivasertib, or a pharmaceutically acceptable salt thereof, is administered at a dosage from about 150 mg to about 250 mg twice daily. In another embodiment, capivasertib, or a pharmaceutically acceptable salt thereof, is administered at a dosage from about 250 mg to about 350 mg twice daily. In another embodiment, capivasertib, or a pharmaceutically acceptable salt thereof, is administered at a dosage from about 350 mg to about 450 mg twice daily. In another embodiment, capivasertib, or a pharmaceutically acceptable salt thereof, is administered at a dosage from about 450 mg to about 550 mg twice daily. In another embodiment, capivasertib, or a pharmaceutically acceptable salt thereof, is administered at a dosage from about 550 mg to about 650 mg twice daily. In another embodiment, capivasertib, or a pharmaceutically acceptable salt thereof, is administered at a dosage from about 650 mg to about 750 mg twice daily. In another embodiment, capivasertib, or a pharmaceutically acceptable salt thereof, is administered at a dosage from about 750 mg to about 850 mg twice daily. In another embodiment, capivasertib, or a pharmaceutically acceptable salt thereof, is administered at a dosage of about 160 mg twice daily. In another embodiment, capivasertib, or a pharmaceutically acceptable salt thereof, is administered at a dosage of about 200 mg twice daily. In another embodiment, capivasertib, or a pharmaceutically acceptable salt thereof, is administered at a dosage of about 240 mg twice daily. In another embodiment, capivasertib, or a pharmaceutically acceptable salt thereof, is administered at a dosage of about 280 mg twice daily. In another embodiment, capivasertib, or a pharmaceutically acceptable salt thereof, is administered at a dosage of about 320 mg twice daily. In another embodiment, capivasertib, or a pharmaceutically acceptable salt thereof, is administered at a dosage of about 360 mg twice daily. In another embodiment, capivasertib, or a pharmaceutically acceptable salt thereof, is administered at a dosage of about 400 mg twice daily. In another embodiment, capivasertib, or a pharmaceutically acceptable salt thereof, is administered at a dosage of about 440 mg twice daily. In another embodiment, capivasertib, or a pharmaceutically acceptable salt thereof, is administered at a dosage of about 480 mg twice daily. In another embodiment, capivasertib, or a pharmaceutically acceptable salt thereof, is administered at a dosage of about 520 mg twice daily. In another embodiment, capivasertib, or a pharmaceutically acceptable salt thereof, is administered at a dosage of about 560 mg twice daily. In another embodiment, capivasertib, or a pharmaceutically acceptable salt thereof, is administered at a dosage of about 600 mg twice daily. In another embodiment, capivasertib, or a pharmaceutically acceptable salt thereof, is administered at a dosage of about 640 mg twice daily. In another embodiment, capivasertib, or a pharmaceutically acceptable salt thereof, is administered at a dosage of about 680 mg twice daily. In another embodiment, capivasertib, or a pharmaceutically acceptable salt thereof, is administered at a dosage of about 720 mg twice daily. In another embodiment, capivasertib, or a pharmaceutically acceptable salt thereof, is administered at a dosage of about 760 mg twice daily. In another embodiment, capivasertib, or a pharmaceutically acceptable salt thereof, is administered at a dosage of about 800 mg twice daily.

In embodiments capivasertib, or a pharmaceutically acceptable salt thereof, is administered under a continuous dosing schedule. In one embodiment, for example, capivasertib, or a pharmaceutically acceptable salt thereof, is administered for more than 1, 2, 3, 4, 5, 6, 7, 14, 21, 28, 35, 42, 49, or 56 days. In another embodiment, the dosing cycle is 28 days. Administration of capivasertib, or a pharmaceutically acceptable salt thereof, and repeat of the dosing cycle can continue as long as tolerable and beneficial for the subject.

In embodiments capivasertib, or a pharmaceutically acceptable salt thereof, is administered once daily (QD) under a continuous dosing schedule. In embodiments capivasertib, or a pharmaceutically acceptable salt thereof, is administered once daily under a continuous dosing schedule at a dosage from about 100 mg to about 900 mg. In another embodiment, capivasertib, or a pharmaceutically acceptable salt thereof, is administered once daily under a continuous dosing schedule at a dosage from about 150 mg to about 875 mg. In another embodiment, capivasertib, or a pharmaceutically acceptable salt thereof, is administered once daily under a continuous dosing schedule at a dosage from about 175 mg to about 850 mg. In another embodiment, capivasertib, or a pharmaceutically acceptable salt thereof, is administered once daily under a continuous dosing schedule at a dosage from about 200 mg to about 825 mg. In another embodiment, capivasertib, or a pharmaceutically acceptable salt thereof, is administered once daily under a continuous dosing schedule at a dosage from about 225 mg to about 800 mg. In another embodiment, capivasertib, or a pharmaceutically acceptable salt thereof, is administered once daily under a continuous dosing schedule at a dosage from about 250 mg to about 750 mg. In another embodiment, capivasertib, or a pharmaceutically acceptable salt thereof, is administered once daily under a continuous dosing schedule at a dosage from about 275 mg to about 700 mg. In another embodiment, capivasertib, or a pharmaceutically acceptable salt thereof, is administered once daily under a continuous dosing schedule at a dosage from about 300 mg to about 650 mg. In embodiments capivasertib, or a pharmaceutically acceptable salt thereof, is administered twice daily (BID) under a continuous dosing schedule. In embodiments capivasertib, or a pharmaceutically acceptable salt thereof, is administered under a continuous dosing schedule at a dosage from about 100 mg to about 800 mg twice daily. In another embodiment, capivasertib, or a pharmaceutically acceptable salt thereof, is administered under a continuous dosing schedule at a dosage from about 150 mg to about 750 mg twice daily. In another embodiment, capivasertib, or a pharmaceutically acceptable salt thereof, is administered under a continuous dosing schedule at a dosage from about 200 mg to about 700 mg twice daily. In another embodiment, capivasertib, or a pharmaceutically acceptable salt thereof, is administered under a continuous dosing schedule at a dosage from about 225 mg to about 650 mg twice daily. In another embodiment, capivasertib, or a pharmaceutically acceptable salt thereof, is administered under a continuous dosing schedule at a dosage from about 250 mg to about 650 mg twice daily. In another embodiment, capivasertib, or a pharmaceutically acceptable salt thereof, is administered under a continuous dosing schedule at a dosage from about 300 mg to about 600 mg twice daily. In another embodiment, capivasertib, or a pharmaceutically acceptable salt thereof, is administered under a continuous dosing schedule at a dosage from about 200 mg to about 300 mg twice daily. In another embodiment, capivasertib, or a pharmaceutically acceptable salt thereof, is administered under a continuous dosing schedule at a dosage from about 300 mg to about 400 mg twice daily. In another embodiment, capivasertib, or a pharmaceutically acceptable salt thereof, is administered under a continuous dosing schedule at a dosage from about 400 mg to about 500 mg twice daily. In another embodiment, capivasertib, or a pharmaceutically acceptable salt thereof, is administered under a continuous dosing schedule at a dosage from about 500 mg to about 600 mg twice daily. In another embodiment, capivasertib, or a pharmaceutically acceptable salt thereof, is administered under a continuous dosing schedule at a dosage from about 600 mg to about 700 mg twice daily. In another embodiment, capivasertib, or a pharmaceutically acceptable salt thereof, is administered under a continuous dosing schedule at a dosage from about 700 mg to about 800 mg twice daily. In another embodiment, capivasertib, or a pharmaceutically acceptable salt thereof, is administered under a continuous dosing schedule at a dosage of about 160 mg twice daily. In another embodiment, capivasertib, or a pharmaceutically acceptable salt thereof, is administered under a continuous dosing schedule at a dosage of about 200 mg twice daily. In another embodiment, capivasertib, or a pharmaceutically acceptable salt thereof, is administered under a continuous dosing schedule at a dosage of about 240 mg twice daily. In another embodiment, capivasertib, or a pharmaceutically acceptable salt thereof, is administered under a continuous dosing schedule at a dosage of about 280 mg twice daily. In another embodiment, capivasertib, or a pharmaceutically acceptable salt thereof, is administered under a continuous dosing schedule at a dosage of about 320 mg twice daily. In another embodiment, capivasertib, or a pharmaceutically acceptable salt thereof, is administered under a continuous dosing schedule at a dosage of about 360 mg twice daily. In another embodiment, capivasertib, or a pharmaceutically acceptable salt thereof, is administered under a continuous dosing schedule at a dosage of about 400 mg twice daily. In another embodiment, capivasertib, or a pharmaceutically acceptable salt thereof, is administered under a continuous dosing schedule at a dosage of about 440 mg twice daily. In another embodiment, capivasertib, or a pharmaceutically acceptable salt thereof, is administered under a continuous dosing schedule at a dosage of about 480 mg twice daily. In another embodiment, capivasertib, or a pharmaceutically acceptable salt thereof, is administered under a continuous dosing schedule at a dosage of about 520 mg twice daily. In another embodiment, capivasertib, or a pharmaceutically acceptable salt thereof, is administered under a continuous dosing schedule at a dosage of about 580 mg twice daily. In another embodiment, capivasertib, or a pharmaceutically acceptable salt thereof, is administered under a continuous dosing schedule at a dosage of about 600 mg twice daily. In another embodiment, capivasertib, or a pharmaceutically acceptable salt thereof, is administered under a continuous dosing schedule at a dosage of about 640 mg twice daily. In another embodiment, capivasertib, or a pharmaceutically acceptable salt thereof, is administered under a continuous dosing schedule at a dosage of about 680 mg twice daily. In another embodiment, capivasertib, or a pharmaceutically acceptable salt thereof, is administered under a continuous dosing schedule at a dosage of about 720 mg twice daily. In another embodiment, capivasertib, or a pharmaceutically acceptable salt thereof, is administered under a continuous dosing schedule at a dosage of about 760 mg twice daily. In another embodiment, capivasertib, or a pharmaceutically acceptable salt thereof, is administered under a continuous dosing schedule at a dosage of about 800 mg twice daily. In embodiments capivasertib, or a pharmaceutically acceptable salt thereof, is administered to the subject on an intermittent dosage schedule. Administering capivasertib, or a pharmaceutically acceptable salt thereof, on an intermittent dosage schedule can, for example, have greater effectiveness and/or tolerability than on a continuous dosing schedule. In embodiments capivasertib, or a pharmaceutically acceptable salt thereof, is intermittently dosed on a 1 day on/6 days off schedule (i.e., capivasertib, or a pharmaceutically acceptable salt thereof, is administered for one day followed by a six-day holiday). In another embodiment, capivasertib, or a pharmaceutically acceptable salt thereof, is intermittently dosed on a 2 days on/5 days off schedule (i.e., capivasertib, or a pharmaceutically acceptable salt thereof, is administered for two days followed by a five-day holiday).

In another embodiment, capivasertib, or a pharmaceutically acceptable salt thereof, is intermittently dosed on a 3 days on/4 days off schedule (i.e., capivasertib, or a pharmaceutically acceptable salt thereof, is administered for three days followed by a four-day holiday). In another embodiment, capivasertib, or a pharmaceutically acceptable salt thereof, is intermittently dosed on a 4 days on/3 days off schedule (i.e., capivasertib, or a pharmaceutically acceptable salt thereof, is administered for four days followed by a three-day holiday). In another embodiment, capivasertib, or a pharmaceutically acceptable salt thereof, is intermittently dosed on a 5 days on/2 days off schedule (i.e., capivasertib, or a pharmaceutically acceptable salt thereof, is administered for five days followed by a two-day holiday).

In another embodiment, capivasertib, or a pharmaceutically acceptable salt thereof, is intermittently dosed on a 6 days on/1 day off schedule (i.e., capivasertib, or a pharmaceutically acceptable salt thereof, is administered for six days followed by a one-day holiday). The dosing cycle of such embodiments would then repeat as long as tolerable and beneficial for the subject. In embodiments the dosing cycle is 7 days. In embodiments the dosing cycle is 14 days. In another embodiment, the dosing cycle is 21 days. In another embodiment, the dosing cycle is 28 days. In another embodiment, the dosing cycle is two months. In another embodiment, the dosing cycle is six months. In another embodiment, the dosing cycle is one year.

In embodiments the dosing cycle is 28 days, but capivasertib, or a pharmaceutically acceptable salt thereof, is not co-administered to the subject during the fourth week of the dosing cycle (i.e., there is a capivasertib, or a pharmaceutically acceptable salt thereof, drug holiday during the final week of the dosing cycle).

In embodiments capivasertib, or a pharmaceutically acceptable salt thereof, is administered once daily (QD) under an intermittent dosing schedule. In embodiments capivasertib, or a pharmaceutically acceptable salt thereof, is administered once daily under an intermittent dosing schedule at a dosage from about 100 mg to about 900 mg. In another embodiment, capivasertib, or a pharmaceutically acceptable salt thereof, is administered once daily under an intermittent dosing schedule at a dosage from about 150 mg to about 850 mg. In another embodiment, capivasertib, or a pharmaceutically acceptable salt thereof, is administered once daily under an intermittent dosing schedule at a dosage from about 175 mg to about 800 mg. In another embodiment, capivasertib, or a pharmaceutically acceptable salt thereof, is administered once daily under an intermittent dosing schedule at a dosage from about 200 mg to about 750 mg. In another embodiment, capivasertib, or a pharmaceutically acceptable salt thereof, is administered once daily under an intermittent dosing schedule at a dosage from about 225 mg to about 725 mg. In another embodiment, capivasertib, or a pharmaceutically acceptable salt thereof, is administered once daily under an intermittent dosing schedule at a dosage from about 250 mg to about 700 mg. In another embodiment, capivasertib, or a pharmaceutically acceptable salt thereof, is administered once daily under an intermittent dosing schedule at a dosage from about 275 mg to about 675 mg. In another embodiment, capivasertib, or a pharmaceutically acceptable salt thereof, is administered once daily under an intermittent dosing schedule at a dosage from about 300 mg to about 650 mg. In embodiments capivasertib, or a pharmaceutically acceptable salt thereof, is administered twice daily (BID) under an intermittent dosing schedule. In embodiments capivasertib, or a pharmaceutically acceptable salt thereof, is administered under an intermittent dosing schedule at a dosage from about 100 mg to about 800 mg twice daily. In another embodiment, capivasertib, or a pharmaceutically acceptable salt thereof, is administered under an intermittent dosing schedule at a dosage from about 150 mg to about 750 mg twice daily. In another embodiment, capivasertib, or a pharmaceutically acceptable salt thereof, is administered under an intermittent dosing schedule at a dosage from about 200 mg to about 700 mg twice daily. In another embodiment, capivasertib, or a pharmaceutically acceptable salt thereof, is administered under an intermittent dosing schedule at a dosage from about 225 mg to about 675 mg twice daily. In another embodiment, capivasertib, or a pharmaceutically acceptable salt thereof, is administered under an intermittent dosing schedule at a dosage from about 250 mg to about 650 mg twice daily. In another embodiment, capivasertib, or a pharmaceutically acceptable salt thereof, is administered under an intermittent dosing schedule at a dosage from about 300 mg to about 625 mg twice daily. In another embodiment, capivasertib, or a pharmaceutically acceptable salt thereof, is administered under an intermittent dosing schedule at a dosage from about 200 mg to about 300 mg twice daily. In another embodiment, capivasertib, or a pharmaceutically acceptable salt thereof, is administered under an intermittent dosing schedule at a dosage from about 300 mg to about 400 mg twice daily. In another embodiment, capivasertib, or a pharmaceutically acceptable salt thereof, is administered under an intermittent dosing schedule at a dosage from about 400 mg to about 500 mg twice daily. In another embodiment, capivasertib, or a pharmaceutically acceptable salt thereof, is administered under an intermittent dosing schedule at a dosage from about 500 mg to about 600 mg twice daily. In another embodiment, capivasertib, or a pharmaceutically acceptable salt thereof, is administered under an intermittent dosing schedule at a dosage from about 600 mg to about 700 mg twice daily. In another embodiment, capivasertib, or a pharmaceutically acceptable salt thereof, is administered under an intermittent dosing schedule at a dosage from about 700 mg to about 800 mg twice daily. In another embodiment, capivasertib, or a pharmaceutically acceptable salt thereof, is administered under an intermittent dosing schedule at a dosage of about 160 mg twice daily. In another embodiment, capivasertib, or a pharmaceutically acceptable salt thereof, is administered under an intermittent dosing schedule at a dosage of about 200 mg twice daily. In another embodiment, capivasertib, or a pharmaceutically acceptable salt thereof, is administered under an intermittent dosing schedule at a dosage of about 240 mg twice daily. In another embodiment, capivasertib, or a pharmaceutically acceptable salt thereof, is administered under an intermittent dosing schedule at a dosage of about 280 mg twice daily. In another embodiment, capivasertib, or a pharmaceutically acceptable salt thereof, is administered under an intermittent dosing schedule at a dosage of about 320 mg twice daily. In another embodiment, capivasertib, or a pharmaceutically acceptable salt thereof, is administered under an intermittent dosing schedule at a dosage of about 360 mg twice daily. In another embodiment, capivasertib, or a pharmaceutically acceptable salt thereof, is administered under an intermittent dosing schedule at a dosage of about 400 mg twice daily. In another embodiment, capivasertib, or a pharmaceutically acceptable salt thereof, is administered under an intermittent dosing schedule at a dosage of about 440 mg twice daily. In another embodiment, capivasertib, or a pharmaceutically acceptable salt thereof, is administered under an intermittent dosing schedule at a dosage of about 480 mg twice daily. In another embodiment, capivasertib, or a pharmaceutically acceptable salt thereof, is administered under an intermittent dosing schedule at a dosage of about 520 mg twice daily. In another embodiment, capivasertib, or a pharmaceutically acceptable salt thereof, is administered under an intermittent dosing schedule at a dosage of about 580 mg twice daily. In another embodiment, capivasertib, or a pharmaceutically acceptable salt thereof, is administered under an intermittent dosing schedule at a dosage of about 600 mg twice daily. In another embodiment, capivasertib, or a pharmaceutically acceptable salt thereof, is administered under an intermittent dosing schedule at a dosage of about 640 mg twice daily. In another embodiment, capivasertib, or a pharmaceutically acceptable salt thereof, is administered under an intermittent dosing schedule at a dosage of about 680 mg twice daily. In another embodiment, capivasertib, or a pharmaceutically acceptable salt thereof, is administered under an intermittent dosing schedule at a dosage of about 720 mg twice daily. In another embodiment, capivasertib, or a pharmaceutically acceptable salt thereof, is administered under an intermittent dosing schedule at a dosage of about 760 mg twice daily. In another embodiment, capivasertib, or a pharmaceutically acceptable salt thereof, is administered under an intermittent dosing schedule at a dosage of about 800 mg twice daily.

In embodiments there is provided a kit comprising a pharmaceutical composition comprising camizestrant and instructions for its use in the treatment of ER+ breast cancer, wherein the use is in combination with capivasertib, optionally where in the use is in further combination with a CDK4/6 inhibitor. In such embodiments, the instructions may direct that the pharmaceutical composition is for use based on the presence of a mutation in PI3KCA or AKT1 or the cancer having been identified as PTEN-deficient.

In embodiments there is provided a kit comprising a pharmaceutical composition comprising capivasertib and instructions for its use in the treatment of ER+ breast cancer, wherein the use is in combination with camizestrant, optionally where in the use is in further combination with a CDK4/6 inhibitor. In such embodiments, the instructions may direct that the pharmaceutical composition is for use based on the presence of a mutation in PI3KCA or AKT1 or the cancer having been identified as PTEN-deficient.

In any embodiment where a marketed or approved drug is mentioned, the marketed or approved drug may be administered in accordance with its label (for example as approved by the United States FDA or any other similar regulatory agency).

In any embodiment where a drug that is being investigated in human trials is mentioned, the drug may be administered in accordance with the dosage regime described in any of its published clinical trial protocols (for example as described on clinicaltrials.gov or similar).

### EXAMPLES

The specific Examples below, with reference to the accompanying Figures, are provided for illustrative purposes only and are not to be construed as limiting the teachings herein.

The text accompanying the figures explains how experiments were run. Cells used in the experiments are discussed below.

MCF7 is a cell line derived from pleural fluid/effusion obtained from a human patient with breast ductal carcinoma. The cell line was obtained from ATCC HTB-22 and harbours the activating mutation in PIK3CA E545K. MCF7 cells were routinely cultured in RPMI (Gibco #11835-063)+5% FCS+1% L-glutamine and incubated at 37° C., 5% CO₂.

MCF7 PC1 cell line was generated from MCF-7 cells cultured in increasing concentrations of palbociclib over the course of 4-9 months until they were able to grow in 1000 nM palbociclib in same cell culture conditions as describe above for parental MCF7.

MCF7 PC6 cell line was generated from MCF-7 cells cultured in increasing concentrations of palbociclib over the course of 4-9 months until they were able to grow in 1000 nM palbociclib in same cell culture conditions as describe above for parental MCF7.

MCF7 PC8 cell line was generated MCF-7 cells cultured in increasing concentrations of palbociclib over the course of 4-9 months until they were able to grow in 1000 nM palbociclib in same cell culture conditions as describe above for parental MCF7.

MCF7 PC10 cell line was generated MCF-7 cells cultured in increasing concentrations of palbociclib over the course of 4-9 months until they were able to grow in 1000 nM palbociclib in same cell culture conditions as describe above for parental MCF7.

All cell lines were generated from the parental ATCC HTB-133 stock with prolonged exposure to fulvestrant and palbociclib. Stock of cells were cultured in T175 flask before starting treatments. Media was removed from flasks, cells were washed with 10 ml DPBS and 2 ml trypsin added to detach them. Once detached, cells were re-suspended in 10 ml of growth media and 10 μl of each was mixed with 10 μl Trypan blue and counted using the ThermoFisher Invitrogen Countess. 10 ml was added to 3×T25 flasks at 2.0×10⁴/ml cells, 2 flasks to be dosed with 30 nM fulvestrant+300 nM palbociclib to generate the resistant pools and 1 flask with equivalent % of DMSO as control. Cells were transferred to an incubator to adhere overnight. Cells were initially dosed with 30 nM fulvestrant & 300 nM palbociclib with the aim to escalate to 100 nM fulvestrant+1 μM palbociclib once the cells start to grow out. Media in the flask was removed and replaced with 10 ml of the fulvestrant+palbociclib containing media (fulvestrant: 2.2 μl 300 μM fulvestrant stock was added to 22 ml growth media, 1:10,000 dilution to give 30 nM final/palbociclib: 2.2 μl 3 mM stock was added to 22 ml growth media, 1:10,000 dilution to give 300 nM final). Cells were re-dosed twice weekly and kept for 6 months as they grew slowly from a small surviving fraction. Cells were expanded to T75 flask and dose escalated to 100 nM fulvestrant+1 μM palbociclib. Cells were expanded for the subsequent 40 days to generate stock for cryopreservation.

CTC-174 is a patient-derived xenograft representative of metastatic breast cancer with ESR1 D538G and PI3KCA N345K mutations (Ladd et al. Oncotarget, 7 (34): 54120-54136, 2016).

ST1799/HI/PBR is a ER+ breast cancer patient derived xenograft tumor model, derived from primary tumor sample with PI3KCA_E542K mutation (provided by XenoSTART).

ST3632 is a ER+ breast cancer patient derived xenograft tumor model with AKT1 mutation (provided by XenoSTART).

ST3932 is a ER+ breast cancer patient derived xenograft tumor model, derived from primary patient samples with PI3KCA_R88Q mutants (provided by XenoSTART).

CTG2432 is a ER+ breast cancer patient derived xenograft tumor model, derived from primary patient samples with ESR1 E380Q and PI3KCA N345K (provided by Champions Oncology).

ST3164B/PBR is a ER+ breast cancer patient derived xenograft tumor model, derived from metastatic patient samples with ESR1_CCDC170 fusion (provided by XenoSTART).

ST941/HI/PBR is a ER+ breast cancer patient derived xenograft tumor model, derived from metastatic patient samples with ESR1 Y537S activation mutation (provided by XenoSTART).

CTG1211 is a ER+ breast cancer patient derived xenograft tumor model, derived from primary patient samples with ESR1 D538G activating mutation (provided by Champions Oncology).

### Example 1: Palbociclib-Resistant Cell Line Combination Experiments

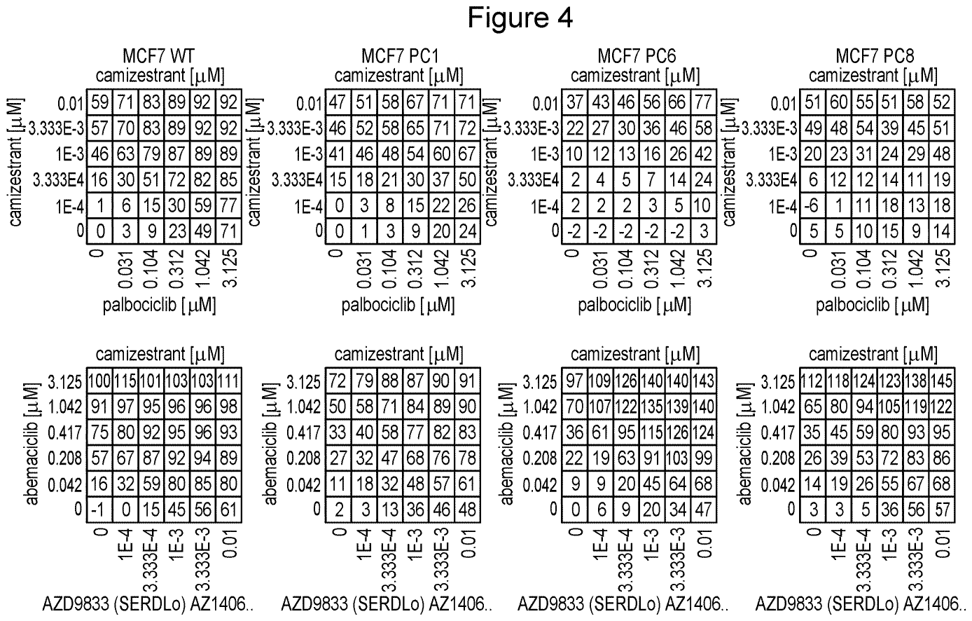

The Highest Single Agent (HSA) model calculates the synergy score matrix for a block of drug combinations. Scores in MCF7 and T47D parental cell lines and palbociclib resistant variants exposed to combination of camizestrant and AZD5363 or everolimus, abemaciclib and palbociclib for 7 days were determined according to the methods described in FIG. 4, and the results shown in Table 1 below. Table 2 summarises the genetic characteristics of the cell lines tested.

TABLE 1
HSA Scores for Camizestrant Combinations

| | MCF7 | MCF7 | MCF7 | MCF7 | MCF7 | T47D | T47D | T47D |
|---|---|---|---|---|---|---|---|---|
| AZD9833+ Compound | wt | PC1 | PC6 | PC8 | PC10 | wt | P1 | P2 |
| Palbociclib (CDK4/6 inhibitor) | 5.9 | 2.4 | 1.8 | 0.5 | 1.7 | 1.9 | 0.1 | 0.6 |
| Abemaciclib (CDK4/6 inhibitor | 5.3 | 4.7 | 15.0 | 7.0 | 6.5 | 1.8 | 0.4 | 0.4 |
| AZD5363 (AKT inhibitor) | 7.5 | 2.0 | 1.9 | 10.1 | 2.2 | 5.0 | 3.8 | 6.4 |
| Everolimus (mTOR inhibitor) | 7.79 | 6.11 | 18.5 | 15.5 | 12.4 | 6.18 | 2.4 | 3.5 |

TABLE 2
Cell genetic characteristics

| Cell line | Genetic background |
|---|---|
| MCF7 | PIK3CA mut, ATM del, BCL2 del, MCL1 amp |
| PC1 | Cdc6, Cyclin D1 and Cyclin E overexpression |
| PC6 | |
| PC8 | Rb Loss, Cdc6 |
| PC10 | |
| T47D | PIK3CA mut |
| T47D P1 | Rb Loss, CCNE1 overexpression |
| T47D P2 | CDK6, CCNE1 overexpression |

### Example 2: Xenograft Experiments

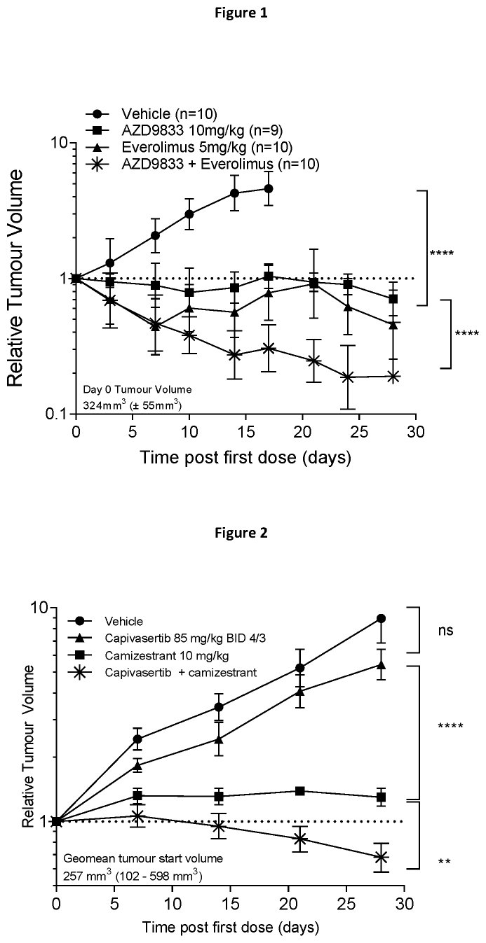

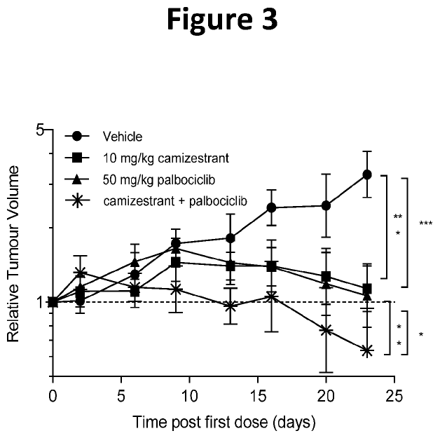

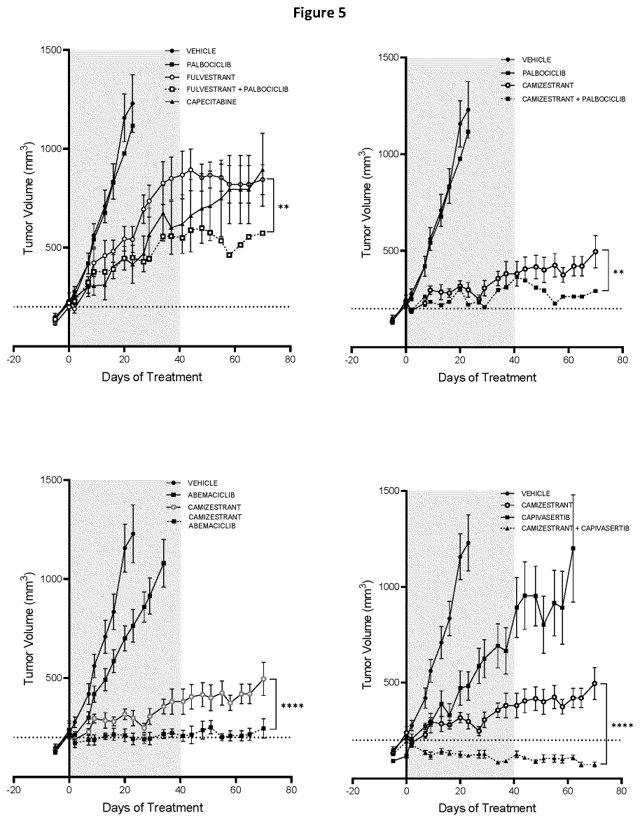

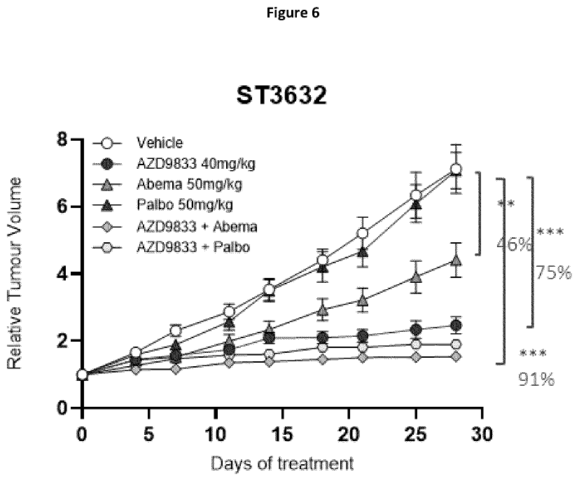

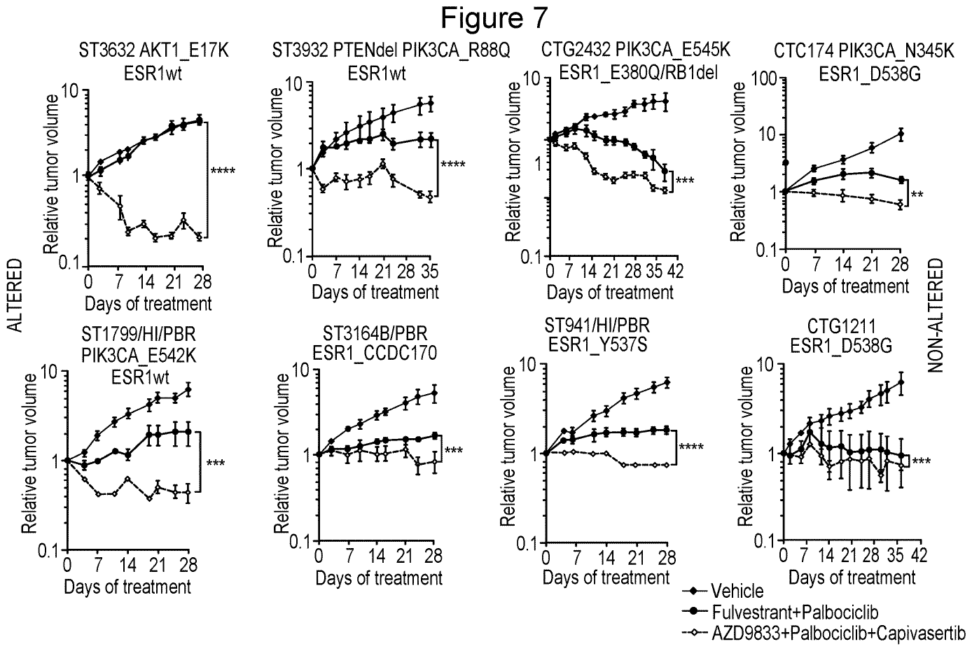

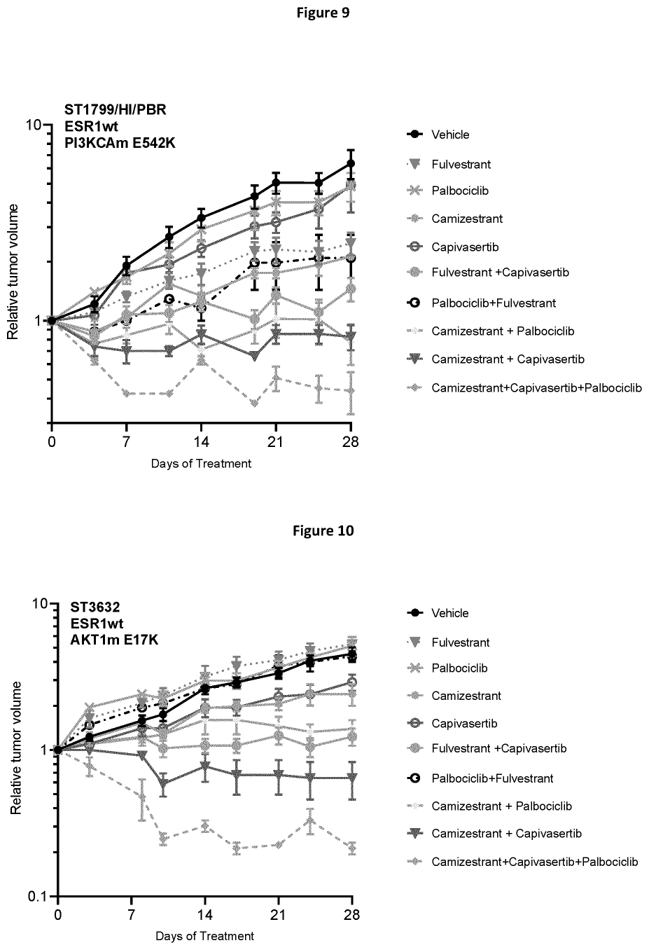

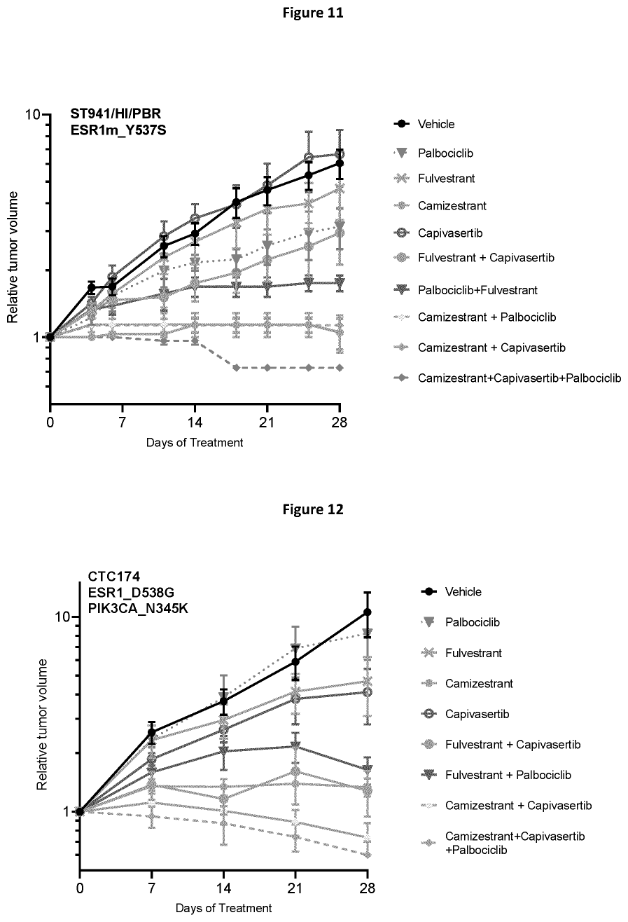

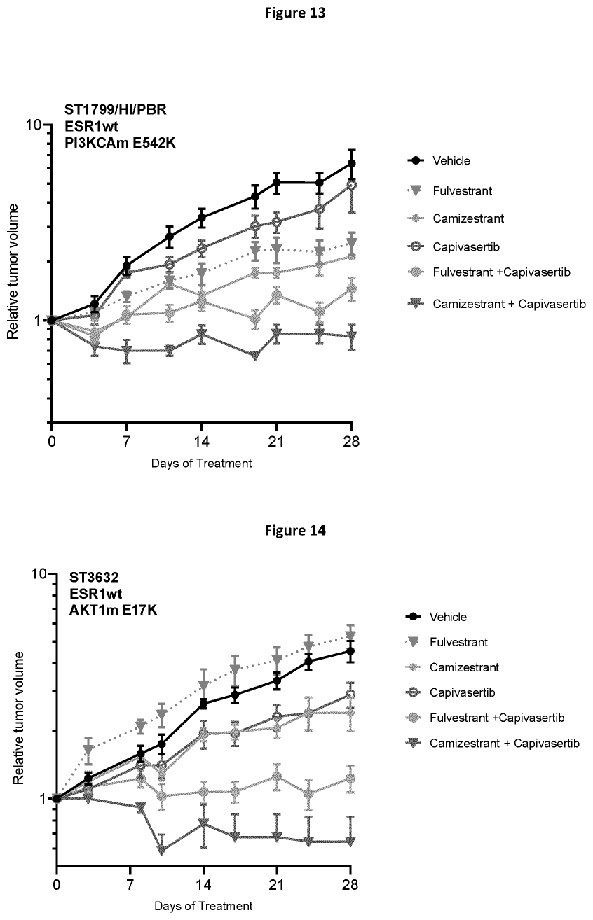

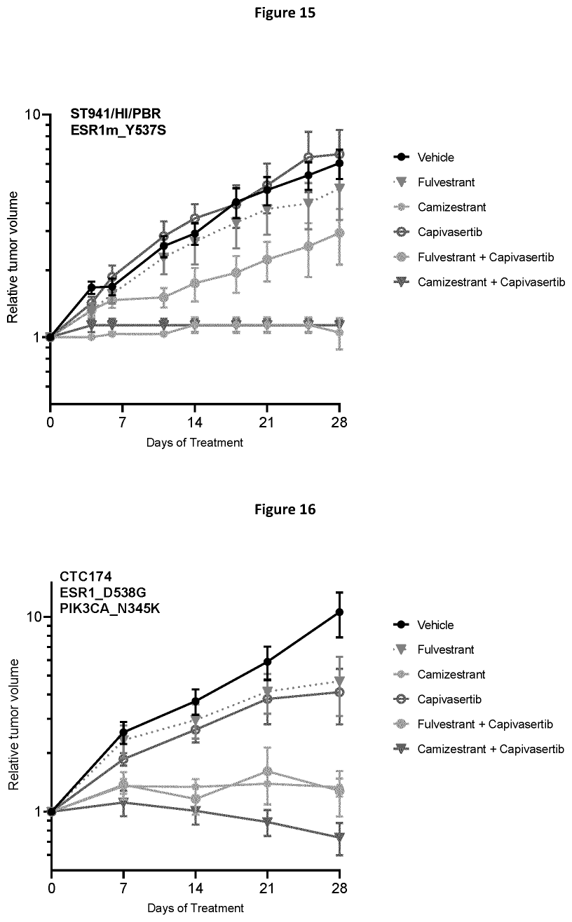

Patient-derived xenograft models were generated from patient biopsies from either metastatic or primary tumors. The samples were implanted in immunocompromised mice for expansion and drug treatment using standard techniques well known in the art. The results of various combination treatments of xenografts are shown in FIGS. 1-3 and 5-16, and are further described in the Figure list.

### Example 3: Clinical Data for Combination of Camizestrant and Capivasertib

### Example 3: Clinical Data for Combination of Camizestrant and Capivasertib

The therapeutic combination of camizestrant and capivasertib was evaluated in Parts I and J of the SERENA-1 study (NCT03616587, see https://classic.clinicaltrials.gov/ct2/show/NCT03616587), a first in human, open label, Phase I study of camizestrant in women with endocrine-resistant ER+, HER2− breast cancer that is not amenable to treatment with curative intent.

In the parts of the SERENA-1 trial in which camizestrant and capivasertib were used in combination, a once daily, 75 mg, oral dose of camizestrant (tablet) was used in conjunction with capivasertib 400 mg dosed twice daily (BID; intermittent; 4 days on, 3 days off) (tablet form). In other words, over the course of each week of treatment a dose of 400 mg of capivasertib was administered twice daily on days 1, 2, 3 and 4, while on days 5, 6 and 7 no capivasertib was administered. Camizestrant meanwhile was dosed once daily at a dose 75 mg on every day of the week.

Demography data for the study participants receiving camizestrant and capivasertib are presented in Table 3. Interim results of the ongoing SERENA-1 study obtained on 14 Sep. 2023 are presented in Table 4.

TABLE 3
Demography of SERENA-1 study participants that
received camizestrant in combination with capivasertib
Demography

| | |
|---|---|
| N | 29 |
| Median age, years (range) | 53 (39-82) |
| Post-menopausal, n (%) | 27 (93%) |
| ECOG category 0, n (%) | 13 (45%) |
| Measurable disease, n (%) | 27 (93%) |
| Visceral disease, n (%) | 21 (72%) |
| Liver Visceral disease, n (%) [a] | 16 (55%) |
| Number of prior regimens in advanced setting, median (range) | 0 (0-3) |
| Number of prior chemotherapy regimens in advanced setting, | 2 (0-5) |
| median (range) | |
| Number of prior endocrine regimens in advanced setting, median | 2 (1-6) |
| (range) | |
| Prior chemotherapy in advanced setting, n (%) | 14 (48%) |
| Prior treatment with fulvestrant in advanced setting, n (%) | 16 (55%) |
| Prior treatment with CDK4/6i in advanced setting, n (%) | 26 (90%) |
| Immediately prior treatment with CDK4/6i, n (%) [b] | 11 (42%) |
| ESR1 ctDNA baseline status, n (%) | |
| ESR1m Not Detected | 12 (41%) |
| ESR1m Detected | 17 (59%) |

The primary objective was to determine the safety and tolerability of camizestrant 75 mg once daily (QD) in combination with capivasertib 400 mg twice daily (BID; intermittent; 4 days on, 3 days off). Secondary objectives included investigation of anti-tumor response and pharmacokinetics (PK). Participants were women of any menopausal status (pre-menopausal women received this combination alongside ongoing ovarian function suppression). Prior treatment with ≤2 lines of chemotherapy in the advanced setting was permitted. Prior treatment with an endocrine therapy (ET) in the advanced setting was required with no limit on the number of lines of prior ET; previous treatment with CDK4/6 inhibitors (CDK4/6i) and fulvestrant was permitted.

### Results:

As of 14 Sep. 2023, 29 patients in Parts I and J of the SERENA-1 study had received camizestrant in combination with capivasertib. As the skilled reader will appreciate, as is usually the case with Phase 1 clinical trials, this Phase 1 study is not powered to provide definitive proof of clinical efficacy or to conclusively demonstrate superiority of one trial arm over another. Nonetheless the results obtained from the study do support the proposition that the promising pre-clinical activity of combinations comprising a ngSERD, such as camizestrant, and an AKT inhibitor, such as capivasertib, described above, can translate successfully into the clinical setting.

The safety and tolerability profile of the combination of camizestrant and capivasertib was broadly consistent with that observed with each drug individually, with no apparent exacerbation of the known tolerability profile for each of the agents. This first in human data for the combination augers well for future clinical use from the safety and tolerability perspective.

Of the heavily pre-treated patients receiving treatment with the combination of camizestrant and capivasertib in the SERENA-1 trial (48% prior chemotherapy, 90% prior CDK4/6i, 55% prior fulvestrant; all in the advanced disease setting) 72% had visceral metastases. In addition, 17 patients had a detectable ESR1m (a mutation or mutations to the gene encoding the estrogen receptor) at baseline and an evaluable C2D1 result. Of these, ESR1m was reduced >50% at C2D1 in 11 cases (91.7%) and, in 8 cases (66.7%) ESR1m was observed to be cleared at C2D1. As can be seen from Table 4, the objective response rate (ORR) observed in the camizestrant/capivasertib combination parts was 37.0% ( 10/29), the clinical benefit rate at 24 weeks (CBR24) was 51.7% ( 15/29) and the median progression-free survival (PFS) was 8.5 months ( 15/29, 95% CI). In patients that had a detectable ESR1m at baseline median PFS was 13.8 months.

The PK and safety data in Parts I and J of SERENA-1 indicated no clinically relevant drug-drug interactions affecting either camizestrant or capivasertib.

The preclinical promise of combinations comprising camizestrant and capivasertib as a novel treatment for ER+ HER2− breast cancer is thus supported by the positive results obtained in a heavily pre-treated patient cohort, including those patients whose tumours have progressed following treatment with CDK4/6 inhibitors, fulvestrant and those with tumours having detectable ESR1m.

TABLE 4
Interim results of SERENA-1 trial as of 14 Sep. 2023

| | Camizestrant + |
|---|---|
| | Capivasertib |
| | (N = 29)^{§} |
| ORR n/m (%)* | 10/29 |
| | (34.5%) |
| ESR1m detected: ORR n/m (%)* | 6/17 |
| | (35.3%) |
| ESR1m not detected: ORR n/m (%) | 4/12 |
| | (33.3%) |
| CBR24 n/m (%)** | 15/29 |
| | (51.7%) |
| ESR1m detected: CBR24 n/m (%)** | 9/17 |
| | (52.9%) |
| ESR1m not detected: CBR24 n/m (%)** | 6/12 |
| | (50%) |
| Median PFS in Months (95% CI) [Maturity] | 8.5 |
| | (4.7, 16.4) |
| | [17/29] |
| ESR1m detected: Median PFS in Months (95% CI) | 13.8 |
| [Maturity] | (4.7, NE) |
| | [8/17] |
| ESR1m not detected: Median PFS in Months | 8.1 |
| (95% CI) [Maturity] | (1.7, 10.9) |
| | [9/12] |

Maturity is the ratio of patients with uncensored PFS over total number evaluated
^{§}once daily oral dose of camizestrant, capivasertib dose and dosing schedule is as described above
*n, number of patients with a CR or PR within m, number of patients in the relevant patient population.
**n, number of patients who have confirmed response or SD for >=23 weeks post-treatment, within m, number of patients who have at least (DCO) date - date of first dose) >=24 weeks or >=23 weeks post-treatment scan.

1-50. (canceled)

51. A method of treating estrogen receptor positive breast cancer in a human patient in need thereof, comprising administering to the human patient a therapeutically effective amount of camizestrant in combination with an AKT inhibitor, and wherein the estrogen receptor positive breast cancer has a PI3KCA mutation or an AKT mutation.

52. The method of treatment according to claim 51, wherein the AKT inhibitor is chosen from miransertib or a pharmaceutically acceptable salt thereof, BAY1125976 or a pharmaceutically acceptable salt thereof, borussertib or a pharmaceutically acceptable salt thereof, AT7867 or a pharmaceutically acceptable salt thereof, CCT128930 or a pharmaceutically acceptable salt thereof, A-674563 or a pharmaceutically acceptable salt thereof, PHT-427 or a pharmaceutically acceptable salt thereof, Akti-1/2 or a pharmaceutically acceptable salt thereof, AT13148 or a pharmaceutically acceptable salt thereof, SC79 or a pharmaceutically acceptable salt thereof, capivasertib or a pharmaceutically acceptable salt thereof, miltefosine or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, afuresertib or a pharmaceutically acceptable salt thereof, uprosertib or a pharmaceutically acceptable salt thereof, XL-418 or a pharmaceutically acceptable salt thereof and ipatasertib or a pharmaceutically acceptable salt thereof.

53. The method of treatment according to claim 52, wherein the AKT inhibitor is capivasertib or a pharmaceutically acceptable salt thereof.

54. The method of treatment according to claim 51, wherein camizestrant is administered in combination with an AKT inhibitor and a CDK4/6 inhibitor chosen from palbociclib or a pharmaceutically acceptable salt thereof, ribociclib or a pharmaceutically acceptable salt thereof and abemaciclib or a pharmaceutically acceptable salt thereof.

55. The method of treatment according to claim 51, wherein where the estrogen receptor positive breast cancer is resistant to treatment with a SERD, a SERM or an aromatase inhibitor or has progressed during or after previous treatment with a SERD, a SERM and/or an aromatase inhibitor.

56. The method of treatment according to claim 51, wherein where the estrogen receptor positive breast cancer is PTEN deficient.

57. The method of treatment according to claim 51, wherein where the estrogen receptor positive breast cancer is resistant to treatment with a CDK4/6 inhibitor or where the cancer has progressed during or after previous treatment with a CDK4/6 inhibitor.

58. The method of treatment according to claim 51, wherein where the estrogen receptor positive breast cancer is CCNE1 amplified, RB1 deficient, overexpresses CDC6 and/or overexpresses CDK6.

59. The method of treatment according to claim 51, wherein where the estrogen receptor positive breast cancer comprises mutant estrogen receptors.

60. The method of treatment according to claim 51, wherein where the estrogen receptor positive breast cancer comprises a AKT mutation and wherein the AKT mutation is an E17K mutation.

61. The method of treatment according to claim 51, wherein where the estrogen receptor positive breast cancer comprises a PI3KCA mutation and wherein the PI3KCA mutation is chosen from R88Q, N345K, C420R, E542K, E545A, E545D, E545Q, E545K, E545G, Q546E, Q546K, Q546R, Q546P, M1043V, M1043I, H1047Y, H1047R, H1047L and G1049R.

62. The method of treatment according to claim 51, wherein patient with estrogen receptor positive breast cancer has not previously been treated with a CDK4/6 inhibitor.

63. The method of treatment according to claim 51, wherein camizestrant 1S administered in combination with and AKT inhibitor and abemaciclib, and the estrogen receptor positive breast cancer is resistant to treatment with palbociclib.
